(19) United States
(12) Patent Application Publication
(10) Pub. No.: US 2025/0304620 A1
(43) Pub. Date: Oct. 2, 2025

(54) ANTIMICROBIAL PEPTIDE AND APPLICATION THEREOF

(71) Applicant: CHINA PHARMACEUTICAL UNIVERSITY, Jiangsu (CN)

(72) Inventor: Miaomiao NIU, Jiangsu (CN)

(73) Assignee: CHINA PHARMACEUTICAL UNIVERSITY, Jiangsu (CN)

(21) Appl. No.: 19/192,339

(22) Filed: Apr. 29, 2025

Related U.S. Application Data
(63) Continuation of application No. PCT/CN2023/143264, filed on Dec. 29, 2023.

(30) Foreign Application Priority Data
Dec. 28, 2023 (CN) 202311824139.3

(51) Int. Cl. C07K 7/06; A61K 9/00; A61K 38/00; A61K 47/60; A61P 31/04; C07K 7/08

(52) U.S. Cl. CPC C07K 7/06; A61K 9/0014; A61K 9/0019; A61K 9/0053; A61K 47/60; A61P 31/04; C07K 7/08; A61K 38/00

(57) ABSTRACT

Disclosed are an antimicrobial peptide, a pharmaceutical composition and application thereof. The antimicrobial peptide (AMP) described in the present disclosure is a highly effective antimicrobial peptide, which has the characteristics of low molecular weight, broad antibacterial spectrum, high efficacy, non-cytotoxicity, non-hemolysis, and high stability, and a low propensity for inducing drug resistance. In addition, they are capable of killing various microorganisms, including clinically common drug-resistant bacterial strains, bacteria and fungi. Furthermore, they have simple structures, are easily synthesized on a large scale and are cost-effective. Therefore, they are expected to replace conventional antibiotics and become a safe, green, efficient and ideal antimicrobial agent, and have promising application prospects in the preparation of antimicrobial drugs, antimicrobial formulations, and antimicrobial products.

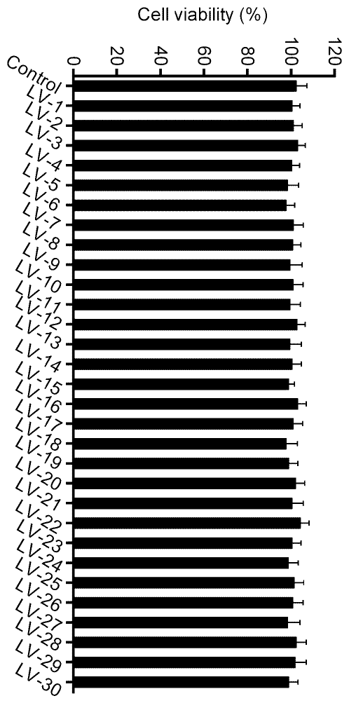

### CROSS-REFERENCE TO RELATED APPLICATION

This application is a continuation of international application of PCT application serial no. PCT/CN2023/143264, filed on Dec. 29, 2023, which claims the priority benefit of China application no. 202311824139.3 filed on Dec. 28, 2023. The entirety of each of the above mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Apr. 28, 2025, is named 155886-US_SEQUENCING_LIST and is 103,019 bytes in size.

### TECHNICAL FIELD

The present disclosure belongs to the field of biomedicine technology, and relates to an antimicrobial peptide and application thereof, and particularly relates to a novel antimicrobial peptide and application thereof in controlling microbial infection.

### RELATED ART

Antibiotics play a significant role in the prevention, control and treatment of human diseases. Since penicillin has been used in clinical practice in the 1940s, infectious diseases have been generally effectively treated. However, with the widespread and long-term use of antibiotics, many pathogenic bacteria have continuously mutated and acquired drug-resistant genes, resulting in the emergence of a large number of drug-resistant strains such as Methicillin-resistant Staphylococcus aureus (MRSA), carbapenem-resistant Escherichia coli (CREC), vancomycin-resistant Enterococcus (VRE), carbapenem-resistant Pseudomonas aeruginosa (CRPA), and carbapenem-resistant Klebsiella pneumoniae (CRKP). The drug-resistant strains can become resistant to antibiotics through a variety of mechanisms, such as producing inactivating enzymes, changing target sites of antibiotics, and pumping drugs out of cells with the help of efflux pumps, and these drug-resistant strains not only spread very quickly but are also difficult to treat. The emergence of drug-resistant bacteria has brought great challenges to the treatment of infectious diseases, and has made the infectious diseases become one of the top ten global public health threats. However, the development of new antibiotics is far slower than the emergence rate of drug-resistant strains, and resistance mechanisms of the next-generation resistant bacteria are becoming increasingly complex. Therefore, there is an urgent need to develop novel, effective, and non-resistant antimicrobial drugs to address the issue of antibiotic resistance.

Antimicrobial peptides (AMPs) are an important class of polypeptides and their derivatives with antimicrobial activity, which exhibit good inhibitory activity against a wide range of microorganisms including bacteria, fungi, viruses, and the like. In addition to the antimicrobial effect, AMPs also have broad application prospects in aquaculture, livestock and poultry feed, food preservation, cosmetics, oral care, genetically modified organisms in plants and animals, and other fields. Compared with chemical drugs, AMPs have many advantages, including strong antimicrobial activity, broad antimicrobial spectrum, good thermal stability, and relatively low toxicity. In addition, compared with traditional single-target antibiotics, the membrane-targeting antimicrobial mechanism of AMPs can enable them to act on multiple targets including the cell membranes at the same time, thereby causing properties of cell membranes to keep stable in a short period of time, therefore, it is not easy for bacteria to develop drug resistance. The non-membrane-targeting mechanism participates in inhibiting essential biological processes such as DNA, RNA, and protein synthesis of bacteria by transferring antimicrobial peptides into the cell. Due to their unique antimicrobial mechanisms, AMPs are considered to have promising prospects and potential research and development value in the treatment of infectious diseases, and they are expected to become a new generation of antimicrobial drugs that could replace traditional antibiotics.

AMPs derive from a wide range of sources. Most of the natural AMPs discovered so far are isolated and purified from organisms such as animals, plants, and bacteria. However, many natural AMPs have been identified, they often face challenges such as large molecular weight, low content, poor stability, and high isolation and purification costs. Furthermore, many AMPs exhibit weak antimicrobial activity or may even cause toxic side effects such as hemolysis to cells. In addition, most of the naturally purified AMPs are composed of L-amino acids, which are unstable in vivo and prone to degradation by proteases. For example, an antimicrobial peptide Cbf-K₁₆, isolated from venom of the Bungarus fasciatus, is composed of thirty L-amino acids, which has poor stability in serum, making it easily degraded and inactivated by proteases. Compared with the L-amino acids, AMPs composed of D-amino acids generally exhibit higher proteolytic stability and metabolic stability. Therefore, the incorporation of D-amino acids into AMPs can disrupt their amphipathic spiral structures, thereby reducing potential side effects such as hemolytic activity and cytotoxicity.

### SUMMARY OF INVENTION

Objectives of the present disclosure: In order to solve the problems existing in the treatment of infectious diseases, the present disclosure provides a strategy of replacing antibiotics with D-type antimicrobial peptides (AMPs), aiming to provide a novel antimicrobial peptide by introducing D-amino acids in place of conventional L-amino acids, thereby solving the common problems of clinical drug resistance of antibiotics, as well as easy degradation, low activity and toxic side effects of natural AMPs. Therefore, the present disclosure is of great significance for the research and development of new and effective anti-infective drugs.

Therefore, one technical problem to be solved by the present disclosure is to provide a novel AMP with broad-spectrum antimicrobial activity, low toxicity, high efficacy, and high stability, and not easy to develop drug resistance.

Another technical problem to be solved by the present disclosure is to provide application of the AMP in various fields, such as pharmaceutical production, livestock and poultry farming, aquaculture, fruit and vegetable preservation, production of disinfection and antiseptic products, additive production, agricultural production, and production of health-related products (food, care products, health supplements, feed, cosmetics and the like).

Technical solution: In order to solve the above technical problems, the present disclosure provides an antimicrobial peptide having a common amino acid general formula of: Ac-Arg-Leu-Leu-B-Z-Leu-Z-B-NH₂, or Ac-Arg-Leu-Leu-B-Z-Leu-Z-B-Arg-NH₂, or Ac-Arg-Arg-Leu-Leu-B-Z-Leu-Z-B-Arg-Arg-NH₂, where Ac represents acetylation modification at an N-terminal of the antimicrobial peptide, NH₂ represents amidation modification at a C-terminal of the antimicrobial peptide, B is selected from aromatic amino acid residues, and one or two Zs are selected from arginine residues or lysine residues.

The aromatic amino acid residues refer to amino acid residues containing an aromatic ring in the molecular structure thereof, and mainly include a 3-(2-naphthyl)-alanine residue, a phenylalanine residue and a phenylalanine-derived residue thereof.

One or more Arg residues in the common amino acid general formula are substituted with lys residues.

One or more Leu residues in the common amino acid general formula are substituted with norleucine residues, norvaline residues, homoleucine residues, alanine residues or valine residues.

The AMP includes all L-enantiomers or all D-enantiomers; alternatively, one or more amino acids in the Common amino acid general formula are substituted with L-amino acids or D-amino acids.

Polyethylene glycol (PEG) is conjugated to An N-terminal or a C-terminal of the AMP, or a side-chain amino group of the lys residue.

A molecular weight of the PEG is 200-4000, preferably, the PEG includes PEG 200, PEG 500, PEG 1000, PEG 2000, or PEG 4000.

The present disclosure further provides a pharmaceutical composition, including the aforementioned AMP.

The present disclosure also provides application of the AMP or the pharmaceutical composition thereof in the preparation of a drug for preventing and/or controlling microbial infection.

Preferably, a dosage range of AMP formulation in the present disclosure is 0.001-1000 mg by weight.

The drug includes at least one of the AMP described above, or a pharmaceutically acceptable salt, ester, a solvate, a hydrate or a prodrug thereof, and at least one pharmaceutically acceptable carrier, supplementary material, excipient, diluent, buffer, adjuvant, auxiliary agent, or vehicle. The term “pharmaceutically acceptable” supplementary material refers to substances that are suitable for humans and/or mammals without undue adverse side effects (such as toxicity, irritation, and allergic response), that is, substances having a reasonable benefit/risk ratio. The term “pharmaceutically acceptable carrier” refers to carriers with which a therapeutic agent is administrated, including various excipients and diluents. It refers to pharmaceutical carriers which are not essential active ingredients themselves and are not overly toxic after administration. Suitable carriers are well known to those of ordinary skill in the art. A full discussion of pharmaceutically acceptable carriers can be found in Remington's Pharmaceutical Sciences (Mack Pub. Co., N.J. 1991). In a composition, the pharmaceutically acceptable carrier may contain liquids such as water, saline, glycerol and ethanol. In addition, auxiliary substances such as lubricants, glidants, wetting agents or emulsifiers, and pH buffering substances, or the like, may also be present in the carriers.

The term “pharmaceutically acceptable supplementary material” refers to any suitable pharmaceutically acceptable adjuvant, carrier, diluent, preservative, or the like, used in pharmaceutical formulations. For exemplary purposes only, known adjuvants include, but are not limited to, for example, complete Freund's adjuvant, incomplete Freund's adjuvant, mineral gels such as aluminum hydroxide, surfactants such as lysolecithin, complex polyols, polyanions, peptides, oil emulsions, hydrocarbon emulsions, keyhole limpet hemocyanin, and the like. Known carriers include, but are not limited to, for example, sterile liquids such as water, normal saline, oil, or mixtures of water and oil, where the oil includes peanut oil, soybean oil, mineral oil, sesame oil, and the like. Known diluents include, but are not limited to, water, saline, glucose, ethanol, glycerol, and the like. Known preservatives include, but are not limited to, thiomersal, ethylenediaminetetraacetic acid (EDTA), and the like. The selection of pharmaceutically acceptable supplementary materials may be achieved using techniques known in the art, and those skilled in the art can select suitable pharmaceutically acceptable supplementary materials based on the antimicrobial peptide drug formulation to be prepared according to the prior art. For example, for the preparation of oral liquid formulations (including but not limited to suspension, microemulsion, or multiple emulsion), the selected supplementary materials may include, for example, water, oil, alcohols, flavoring agents, preservatives, coloring agents, and the like. For another example, for the preparation of oral solid formulations (including but not limited to powders, powder aerosols, capsules, or tablets), the selected supplementary materials may include, for example, starch, sugar, diluents, granulating agents, lubricants, binders, disintegrants, and the like. In addition, if needed, the antimicrobial peptide drug of the present disclosure may be prepared into a sugar coating or an enteric coating, or a controlled-release formulation.

A dosage form of the drug may include injectable formulation, tablet, oral formulation, topical formulation, eye drop, lotion, powder, granule, dripping pill, capsule, caplet, pill, powder formulations, elixir, suspension, mixture, enema, liniment, solution, liquid formulation, rubber formulation, mucilage, liquid extract, extract, gel, sachet, flat capsule, solution agent, syrup, aqueous agent, spirit, glycerin formulation, transdermal patch, film-forming agent, instant granule, smoke formulation, emulsion, transdermal plaster, tincture, penetrant, wine formulation, traditional Chinese medicine formulation, aerosol, cream, ear drop, ointment, oral dissolvable film, mouthwash, cleansing solution, paste, spray, foam, inhalant, gel formulation, injection, medicinal distillate, dry powder injection, enteric coating, nanosphere, microsphere, powder aerosol, decoction, plaster, bolus, nasal spray, nasal drop, garle, sublingual tablet, insufflation powder, suppository, film agent, drop formulation, liniment, nasal wash, eye wash, ear wash, intraocular insert, ocular film, ophthalmic ointment, powder spray, dry powder inhalation, powder inhalation, atomized inhalation, stick formulation, thread formulation, strip formulation, tooth drop, suspension drop, latex agent, sprinkle formulation, coating, coating agent, cream, patch, transdermal, oral patch, Dosage forms of the drug may include injectable formulation, tablet, oral formulation, topical formulation, eye drop, lotion, powder, granule, dripping pill, capsule, caplet, pill, powder formulations, elixir, suspension, mixture, enema, liniment, solution, liquid formulation, rubber formulation, mucilage, glue, extract, gel, sachet, flat capsule, solution agent, syrup, aqueous agent, spirit, glycerin formulation, transdermal patch, film-forming agent, instant granule, smoke formulation, emulsion, transdermal plaster, tincture, penetrant, wine formulation, traditional Chinese medicine formulation, aerosol, cream, ear drop, ointment, oral dissolvable film, mouthwash, cleansing solution, paste, spray, foam, inhalant, gel formulation, injection, medicinal distillate, dry powder injection, enteric coating, nanosphere, microsphere, powder aerosol, decoction, plaster, bolus, nasal spray, nasal drop, garle, sublingual tablet, insufflation powder, suppository, film agent, drop formulation, liniment, nasal wash, eye wash, ear wash, intraocular insert, ocular film, ophthalmic ointment, powder spray, dry powder inhalation, powder inhalation, atomized inhalation, stick formulation, thread formulation, strip formulation, tooth drop, suspension drop, latex agent, sprinkle formulation, coating, coating agent, cream, patch, transdermal, oral patch, subdermal, irrigation agent, lozenge, cake formulation, medicinal cake, concentrated decoction, medicated plaster, tea formulation, porridge formulation, oil extract, sol agent, fluid extract, powder for injection, aqueous injection, sterile powder, sponge agent, microcapsule, nanocapsule, honeyed pill, water pill, water-honeyed pill, paste pill, concentrated pill, waxed pill, sugar-coated pill, film-coated tablet, enteric-coated tablet, effervescent tablet, chewable tablet, soluble tablet, dispersible tablet, sustained-release tablet, controlled-release tablet, orally disintegrating tablet, buccal pill, and microemulsion or multiple emulsion.

Preferably, the dosage form of the drug includes an injectable formulation, an oral formulation, or a topical formulation, where the topical formulation includes eye drop or lotion, a dosage range of the AMP in the dosage form is as follows: 0.001-1000 mg/kg for the injectable formulation, 0.001-1000 mg/kg for the oral formulation, 1/10000-30% per tube for the topical formulation, 1/10000-30% per tube for the eye drop, and 1/100000-20% % per tube for the lotion.

The microorganisms include one or more of bacteria, fungi, actinomycetes, archaea, cyanobacteria, mycoplasma, chlamydia, rickettsia, spirochetes, subviruses, viruses, protozoa, or algae.

The application includes any of the following:
(1) application of the AMP or a pharmaceutical composition thereof in the preparation of anti-inflammatory and/or anti-tumor drugs;
(2) application of the AMP or a pharmaceutical composition thereof in aquaculture;
(3) application of the AMP or a pharmaceutical composition thereof in the preparation of oral care products;
(4) application of the AMP or a pharmaceutical composition thereof in the preparation of oral cleansing formulations;
(5) application of the AMP or a pharmaceutical composition thereof in the preparation of tooth cleaning products;
(6) application of the AMP or a pharmaceutical composition thereof in the preparation of dental coatings;
(7) application of the AMP or a pharmaceutical composition thereof in the preparation of drugs for the prevention and treatment of dental caries;
(8) application of the AMP or a pharmaceutical composition thereof in the preparation of products for treating skin infections;
(9) application of the AMP or a pharmaceutical composition thereof in the preparation of intracellular bactericidal products;
(10) application of the AMP or a pharmaceutical composition thereof in the preparation of products for removing bacterial biofilms;
(11) application of the AMP or a pharmaceutical composition thereof in the preparation of disinfectant products;
(12) application of the AMP or a pharmaceutical composition thereof in preventing and/or treating wheat Fusarium head blight;
(13) application of the AMP or a pharmaceutical composition thereof in the preparation of mildew-proof and preservative agents;
(14) application of the AMP or a pharmaceutical composition thereof in the preparation of food preservatives;
(15) application of the AMP or a pharmaceutical composition thereof in the preparation of cosmetic preservatives;
(16) application of the AMP or a pharmaceutical composition thereof in the preparation of cosmetics;
(17) application of the AMP or a pharmaceutical composition thereof in livestock and poultry feed and/or feed additives;
(18) application of the AMP or a pharmaceutical composition thereof in the improvement of animal and plant varieties and/or breeding of animal and plant;
(19) application of the AMP or a pharmaceutical composition thereof in the preparation of wound dressings;
(20) application of the AMP or a pharmaceutical composition thereof in the preparation of antimicrobial preservation films;
(21) application of the AMP or a pharmaceutical composition thereof in the preservation of fruit and vegetable;
(22) application of the AMP or a pharmaceutical composition thereof in livestock farming; and
(23) application of the AMP or a pharmaceutical composition thereof in the preparation of antimicrobial agents, pharmaceuticals, veterinary drugs, feeds, food products, daily chemicals, health care products, cosmetics, bactericides, virucides, algaecides, sterilants, additives, oral cleansing formulations, disinfectants, anti-inflammatory products, detergents, preservatives, supplementary materials, mildew-proof agents, algae-proof agents, detergent auxiliaries, detergent compositions, cleaning agents, or preservatives.

The above-mentioned AMPs and a pharmaceutical composition thereof may be prepared into any medically usable biological carrier or dosage form for administration to patients suffering from infectious diseases.

Beneficial effects: Compared with the prior art, the present disclosure has the following advantages: the AMP described in the present disclosure is a highly effective antimicrobial peptide, which have the characteristics of low molecular weight, broad antibacterial spectrum, high efficacy, non-cytotoxicity, non-hemolysis, and high stability, and a low propensity for inducing drug resistance. In addition, they are capable of killing various microorganisms, including clinically common drug-resistant bacterial strains, bacteria and fungi. Furthermore, they have simple structures, are easily synthesized on a large scale and are cost-effective. Therefore, they are expected to replace conventional antibiotics and become a safe, green, efficient and ideal antimicrobial agent, and have promising application prospects in the preparation of antimicrobial drugs, antimicrobial formulations, and antimicrobial products.

### BRIEF DESCRIPTION OF DRAWINGS

FIGURE illustrates cytotoxicity experimental results of AMPs LV-1 to LV-30 according to the present disclosure.

### DESCRIPTION OF EMBODIMENTS

Unless otherwise defined, the technical terms used in the following examples have the same meaning as those generally understood by those skilled in the art to which the present disclosure belongs. Unless otherwise specified, the test reagents used in the following examples are conventional biochemical reagents, and unless otherwise specified, the experimental methods are conventional techniques.

The present disclosure will be further described below in conjunction with specific examples.

### Example 1 Information of Antimicrobial Peptide Sequences

It can be seen from Table 1 (summary information of antimicrobial peptide sequences) that the first 30 antimicrobial peptides (AMPs) (LV-1 to LV-30) numbered from 1 to 30 are all-D-amino acids; and the last 30 AMPs (LV-31 to LV-60) numbered from 31 to 60 are all-L-amino acids. In Table 1, X represents a 3-(2-naphthyl)-alanine residue, X1 represents a 4-fluorophenylalanine residue, X2 represents a 3,4-difluorophenylalanine residue, X3 represents a 4-chlorophenylalanine residue, X4 represents a 3,4-dichlorophenylalanine residue, X5 represents a 4-(trifluoromethyl-)phenylalanine residue, X6 represents a 4-bromophenylalanine residue, X7 represents a 4-methyl-phenylalanine residue, X8 represents a norleucine residue, X9 represents a norvaline residue, X10 represents a homoleucine residue, Ac represents acetylation modification at an N-terminal of the antimicrobial peptide, and NH₂ represents amidation modification at a C-terminal of the antimicrobial peptide. The phenylalanine-derived residue mainly includes the 4-fluorophenylalanine residue, the 3,4-difluorophenylalanine residue, the 4-chlorophenylalanine residue, the 3,4-dichlorophenylalanine residue, the 4-(trifluoromethyl-)phenylalanine residue, the 4-bromophenylalanine residue, and the 4-methyl-phenylalanine residue. In addition, for the 25 AMPs numbered 1-25 (LV-1 to LV-25) and the 25 AMPs numbered 31-55 (LV-31 to LV-55) listed in Table 1, their N-terminals are subjected to acetylation modification, and their C-terminals are subjected to amidation modification. PEG200 of the AMPs LV-26 and LV-56 is polyethylene glycol 200, where the PEG200 is conjugated to C-terminals of the LV-26 and LV-56, respectively; PEG500 of the AMPs LV-27 and LV-57 is polyethylene glycol 500, where the PEG500 is conjugated to C-terminals of the LV-27 and LV-57, respectively; PEG1000 of the LV-28 and LV-58 is polyethylene glycol 1000, and the PEG 1000 is conjugated to C-terminals of the LV-28 and LV-58, respectively; PEG2000 of the LV-29 and LV-59 is polyethylene glycol 2000, and the PEG2000 is conjugated to N-terminals of the LV-29 and LV-59, respectively; and PEG4000 of the LV-30 and LV-60 is polyethylene glycol 4000, and the PEG4000 is conjugated to C-terminals of the LV-30 and LV-60, respectively.

The information of antimicrobial peptide sequences is summarized in Table 1. All 60 peptides (LV-1 to LV-60) have a common amino acid sequence motif structure, the 1^{st}, 2^{nd}, 6^{th}, 8^{th}, 10^{th}, and 11^{th} amino acids of them are positively charged arginine or lysine, the 3^{rd}, 4^{th}, and 7^{th} amino acids of them are all hydrophobic amino acids, and the 5^{th} and 9^{th} amino acids are all aromatic amino acids. Experiments were performed among all the 60 polypeptides (LV-1 to LV-60) having the common motif structure, and it was found that 10 representative polypeptides (LV-1, LV-2, LV-6, LV-12, LV-17, LV-18, LV-19, LV-20, LV-25, and LV-27) demonstrated significant therapeutic effects when conducting in vivo pharmacodynamic studies in high, medium, and low dose groups.

TABLE 1
Summary of AMP Sequences

| | Name of | Amino acid sequence | | | | | | | | | | |
|---|---|---|---|---|---|---|---|---|---|---|---|---|
| S/N | AMP | 1 | 12 | 3 | 4 | 5 | 6 | 7 | 8 | 9 | 10 | 11 |
| D-enantiomer | | D | D | D | D | D | D | D | D | D | D | D |
| 1 | LV-1 | R | R | L | L | X | R | L | R | X | R | R |
| 2 | LV-2 | R | K | L | L | F | R | L | K | F | R | K |
| 3 | LV-3 | R | K | L | L | F | R | L | K | X | R | K |
| 4 | LV-4 | R | K | L | L | X | R | L | K | F | R | K |
| 5 | LV-5 | R | K | L | L | X1 | K | L | K | X1 | K | R |
| 6 | LV-6 | R | K | L | L | X2 | K | L | K | X2 | K | R |
| 7 | LV-7 | R | K | L | L | X3 | K | L | K | X3 | K | R |
| 8 | LV-8 | R | K | L | L | X4 | K | L | K | X4 | K | R |
| 9 | LV-9 | R | K | L | L | X5 | K | L | K | X5 | K | R |
| 10 | LV-10 | K | K | L | L | X6 | R | L | K | X6 | R | K |
| 11 | LV-11 | R | K | L | L | X7 | K | L | K | X7 | K | R |
| 12 | LV-12 | R | K | X8 | X8 | X | R | X8 | K | X | R | K |
| 13 | LV-13 | R | K | X9 | X9 | X | R | X9 | K | X | R | K |
| 14 | LV-14 | R | K | X10 | X10 | X | R | X10 | K | X | R | K |
| 15 | LV-15 | R | K | A | A | X | R | A | K | X | R | K |
| 16 | LV-16 | R | K | V | V | X | R | V | K | X | R | K |
| 17 | LV-17 | K | K | L | L | X | K | L | K | X | K | K |
| 18 | LV-18 | R | K | L | L | X | K | L | K | X | K | R |
| 19 | LV-19 | K | K | L | L | X | R | L | K | X | R | K |
| 20 | LV-20 | R | K | L | L | X | R | L | K | X | R | K |
| 21 | LV-21 | R | R | L | L | X | R | L | K | X | R | K |
| 22 | LV-22 | R | K | L | L | X | R | L | K | X | R | R |
| 23 | LV-23 | K | R | L | L | X | K | L | K | X | K | R |
| 24 | LV-24 | R | R | L | L | X | K | L | K | X | K | R |
| 25 | LV-25 | K | K | L | L | X2 | R | L | K | X2 | R | K |
| 26 | LV-26 | Ac-RKLLXRLKXRK-PEG200 | | | | | | | | | | |
| 27 | LV-27 | Ac-RKLLXRLKXRK-PEG500 | | | | | | | | | | |
| 28 | LV-28 | Ac-RKLLXRLKXRK-PEG1000 | | | | | | | | | | |
| 29 | LV-29 | PEG2000-RKLLXRLKXRK-NH₂ | | | | | | | | | | |
| 30 | LV-30 | Ac-RKLLXRLKXRK-PEG4000 | | | | | | | | | | |
| | | L | L | L | L | L | L | L | L | L | L | L |
| 31 | LV-31 | R | R | L | L | X | R | L | R | X | R | R |
| 32 | LV-32 | R | K | L | L | F | R | L | K | F | R | K |
| 22 | LV-33 | R | K | L | L | F | R | L | K | X | R | K |
| 34 | LV-34 | R | K | L | L | X | R | L | K | F | R | K |
| 35 | LV-35 | R | K | L | L | X1 | K | L | K | X1 | K | R |
| 36 | LV-36 | R | K | L | L | X2 | K | L | K | X2 | K | R |
| 37 | LV-37 | R | K | L | L | X3 | K | L | K | X3 | K | R |
| 38 | LV-38 | R | K | L | L | X4 | K | L | K | X4 | K | R |
| 39 | LV-39 | R | K | L | L | X5 | K | L | K | X5 | K | R |
| 40 | LV-40 | K | K | L | L | X6 | R | L | K | X6 | R | K |
| 41 | LV-41 | R | K | L | L | X7 | K | L | K | X7 | K | R |
| 42 | LV-42 | R | K | X8 | X8 | X | R | X8 | K | X | R | K |
| 43 | LV-43 | R | K | X9 | X9 | X | R | X9 | K | X | R | K |
| 44 | LV-44 | R | K | X10 | X10 | X | R | X10 | K | X | R | K |
| 45 | LV-45 | R | K | A | A | X | R | A | K | | R | K |
| 46 | LV-46 | R | K | V | V | X | R | V | K | X | R | K |
| 47 | LV-47 | K | K | L | L | X | K | L | K | X | K | K |
| 48 | LV-48 | R | K | L | L | X | K | L | K | X | K | R |
| 49 | LV-49 | K | K | L | L | X | R | L | K | X | R | K |
| 50 | LV-50 | R | K | L | L | X | R | L | K | X | R | K |
| 51 | LV-51 | R | R | L | L | X | R | L | K | X | R | K |
| 52 | LV-52 | R | K | L | L | X | R | L | K | X | R | R |
| 53 | LV-53 | K | R | L | L | X | K | L | K | X | K | R |
| 54 | LV-54 | R | R | L | L | X | K | L | K | X | K | R |
| 55 | LV-55 | K | K | L | L | X2 | R | L | K | X2 | R | K |

| | | |
|---|---|---|
| 56 | LV-56 | Ac-RKLLXRLKXRK-PEG200 |
| 57 | LV-57 | Ac-RKLLXRLKXRK-PEG500 |
| 58 | LV-58 | Ac-RKLLXRLKXRK-PEG1000 |
| 59 | LV-59 | PEG2000-RKLLXRLKXRK-NH₂ |
| 60 | LV-60 | Ac-RKLLXRLKXRK-PEG4000 |

### Example 2: Antibacterial Activity Experiment of AMPs

1. Antibacterial Experiment of LV-1˜LV-60 Against Staphylococcus aureus (NRS 384)

According to the Standards of Clinical and Laboratory Standards Institute (CLSI) of the USA for antimicrobial drug susceptibility test operation standards, a micronutrient serial twofold dilution method was used to determine minimum inhibitory concentrations (MICs) of all 60 peptides (S/N: LV-1˜LV-60) against Staphylococcus aureus (NRS 384). The experiment was repeated 5 times in parallel. Final concentrations of polypeptide solutions were 0.125, 0.25, 0.5, 1, 2, 4, 8, 16, 32, 64, and 128 μg/mL, and a lowest drug concentration that completely inhibited bacterial growth in the wells was identified as the MIC of the AMPs. An antibacterial drug Levofloxacin and an AMP Cbf-K₁₆ (sequence of Cbf-K₁₆: KFFRKLKKSVKKRAKKFFKKPRVIGVSIPF) were used as positive controls to evaluate the antibacterial effect of the antimicrobial peptides on Staphylococcus aureus (NRS 384). Results are shown in Table 2.

As shown in Table 2, compared with the AMP Cbf-K₁₆ (MIC=32 μg/mL), all 60 tested polypeptides (S/N: LV-1˜LV-60) exhibited better antibacterial effect (MIC≥8 μg/mL) on Staphylococcus aureus (NRS 384), among which 30 polypeptides (S/N: LV-1 to LV-30) demonstrated the best antibacterial activity (MIC=1 μg/mL), comparable to that of the antibacterial activity of the antibacterial drug Levofloxacin.

TABLE 2
Experimental results of MIC (μg/mL) of all 60 rested polyeptides
(S/N: LV-1 to LV-60) against Staphylococcus aureus (NRS 384)

| | Staphylococcus |
|---|---|
| S/N | aureus |
| LV-1 | 1 |
| LV-2 | 1 |
| LV-3 | 1 |
| LV-4 | 1 |
| LV-5 | 1 |
| LV-6 | 1 |
| LV-7 | 1 |
| LV-8 | 1 |
| LV-9 | 1 |
| LV-10 | 1 |
| LV-11 | 1 |
| LV-12 | 1 |
| LV-13 | 1 |
| LV-14 | 1 |
| LV-15 | 1 |
| LV-16 | 1 |
| LV-17 | 1 |
| LV-18 | 1 |
| LV-19 | 1 |
| LV-20 | 1 |
| LV-21 | 1 |
| LV-22 | 1 |
| LV-23 | 1 |
| LV-24 | 1 |
| LV-25 | 1 |
| LV-26 | 1 |
| LV-27 | 1 |
| LV-28 | 1 |
| LV-29 | 1 |
| LV-30 | 1 |
| LV-31 | 4 |
| LV-32 | 4 |
| LV-33 | 8 |
| LV-34 | 4 |
| LV-35 | 8 |
| LV-36 | 4 |
| LV-37 | 4 |
| LV-38 | 8 |
| LV-39 | 8 |
| LV-40 | 4 |
| LV-41 | 4 |
| LV-42 | 8 |
| LV-43 | 8 |
| LV-44 | 8 |
| LV-45 | 2 |
| LV-46 | 4 |
| LV-47 | 2 |
| LV-48 | 4 |
| LV-49 | 8 |
| LV-50 | 4 |
| LV-51 | 4 |
| LV-52 | 8 |
| LV-53 | 4 |
| LV-54 | 8 |
| LV-55 | 4 |
| LV-56 | 4 |
| LV-57 | 8 |
| LV-58 | 4 |
| LV-59 | 4 |
| LV-60 | 8 |
| Levofloxacin | 1 |
| Cbf-K₁₆ | 32 |

### 2. Antibacterial Experiment of LV-1˜LV-30 on 10 Clinically Common Bacterial Strains and 6 Fungi in Clinical Practice

According to the Standards of Clinical and Laboratory Standards Institute (CLSI) of the USA for antimicrobial drug susceptibility test operation standards, the micronutrient serial twofold dilution method was used further determine MICs of 30 AMPs (S/N: LV-1 to LV-30) 10 clinically common sensitive strains and 6 fungi in clinical practice. The experiment was repeated 5 times in parallel. Final concentrations of polypeptide solutions were 0.125, 0.25, 0.5, 1, 2, 4, 8, 16, 32, 64, and 128 μg/mL, and a lowest drug concentration that completely inhibited the growth of bacteria or fungi in the wells was identified as the MIC (μg/mL) of the AMPs. Experimental results are shown in Tables 3 and 4.

It can be seen from the experimental results in Tables 3 and 4, LV-1 to LV-30 exhibited strong antimicrobial activity against all the 16 clinically common sensitive strains and fungi tested.

TABLE 3
Experimental results of MICs (μg/mL) of LV-1 to LV-30 against 10 clinically common bacterial strains

| | Staphylococcus | Escherichia | Klebsiella | Salmonella | Pseudomonas | | Bacillus | Coryne- | Shigella | Serratia |
|---|---|---|---|---|---|---|---|---|---|---|
| | aureus | coli | pneumoniae | typhimurium | aeruginosa | Enterococcus | subtilis | bacterium | flexneri | marcescens |
| Strain | ATCC | ATCC | ATCC | ATCC | ATCC | ATCC | ATCC | ATCC | ATCC | ATCC |
| type | 25923 | 25922 | 700603 | 14028 | 27853 | 29212 | 6633 | 7715 | 12022 | 29021 |
| LV-1 | 1 | 1 | 4 | 2 | 1 | 2 | 4 | 2 | 4 | 2 |
| LV-2 | 2 | 1 | 4 | 2 | 1 | 4 | 2 | 4 | 2 | 4 |
| LV-3 | 2 | 1 | 2 | 1 | 4 | 4 | 2 | 4 | 2 | 4 |
| LV-4 | 1 | 2 | 4 | 2 | 2 | 2 | 4 | 2 | 4 | 2 |
| LV-5 | 1 | 4 | 2 | 4 | 2 | 2 | 4 | 4 | 2 | 2 |
| LV-6 | 1 | 1 | 4 | 2 | 1 | 2 | 4 | 2 | 4 | 8 |
| LV-7 | 1 | 4 | 2 | 4 | 2 | 8 | 4 | 4 | 4 | 8 |
| LV-8 | 1 | 2 | 4 | 2 | 2 | 2 | 2 | 2 | 4 | 2 |
| LV-9 | 2 | 2 | 2 | 2 | 2 | 4 | 8 | 2 | 2 | 4 |
| LV-10 | 2 | 1 | 2 | 2 | 2 | 2 | 2 | 8 | 2 | 4 |
| LV-11 | 1 | 2 | 4 | 2 | 2 | 2 | 4 | 8 | 4 | 8 |
| LV-12 | 1 | 1 | 2 | 4 | 1 | 4 | 8 | 2 | 4 | 2 |
| LV-13 | 2 | 2 | 4 | 2 | 4 | 8 | 4 | 4 | 8 | 4 |
| LV-14 | 2 | 2 | 4 | 4 | 4 | 4 | 2 | 8 | 4 | 8 |
| LV-15 | 2 | 2 | 4 | 2 | 4 | 4 | 4 | 8 | 2 | 8 |
| LV-16 | 1 | 1 | 2 | 2 | 4 | 2 | 4 | 2 | 2 | 4 |
| LV-17 | 1 | 1 | 4 | 4 | 1 | 2 | 4 | 2 | 4 | 2 |
| LV-18 | 2 | 1 | 2 | 4 | 1 | 4 | 4 | 2 | 8 | 4 |
| LV-19 | 1 | 1 | 2 | 2 | 1 | 2 | 8 | 2 | 4 | 8 |
| LV-20 | 1 | 1 | 2 | 1 | 1 | 4 | 2 | 2 | 4 | 2 |
| LV-21 | 1 | 2 | 4 | 2 | 2 | 4 | 2 | 4 | 2 | 4 |
| LV-22 | 1 | 2 | 2 | 4 | 2 | 2 | 4 | 4 | 4 | 4 |
| LV-23 | 1 | 2 | 4 | 2 | 2 | 8 | 4 | 4 | 2 | 8 |
| LV-24 | 1 | 1 | 4 | 1 | 2 | 4 | 2 | 4 | 2 | 2 |
| LV-25 | 2 | 1 | 8 | 1 | 1 | 2 | 8 | 2 | 2 | 4 |
| LV-26 | 1 | 2 | 4 | 2 | 4 | 8 | 4 | 2 | 4 | 8 |
| LV-27 | 1 | 1 | 8 | 2 | 1 | 2 | 2 | 8 | 2 | 4 |
| LV-28 | 2 | 2 | 4 | 4 | 2 | 8 | 4 | 8 | 4 | 8 |
| LV-29 | 2 | 1 | 4 | 4 | 4 | 8 | 4 | 16 | 4 | 4 |
| LV-30 | 2 | 2 | 8 | 2 | 2 | 4 | 2 | 8 | 2 | 8 |

TABLE 4
Experiment results of MICs (μg/mL) of LV-1 to LV-30 against common fungi

| | Candida | | | | | |
|---|---|---|---|---|---|---|
| | albicans | Candida | Candida | Candida | Candida | Trichophyton |
| | ATCC | krusei | utilis | tropicalis | glabrata | ATCC |
| Strain | MYA- | ATCC | ATCC | ATCC | ATCC | MYA- |
| type | 2876 | 6258 | 9950 | 750 | 15126 | 4438 |
| LV-1 | 8 | 4 | 8 | 4 | 8 | 8 |
| LV-2 | 16 | 16 | 8 | 16 | 16 | 8 |
| LV-3 | 8 | 16 | 8 | 8 | 16 | 16 |
| LV-4 | 8 | 16 | 8 | 16 | 8 | 8 |
| LV-5 | 8 | 4 | 16 | 8 | 16 | 4 |
| LV-6 | 16 | 4 | 8 | 4 | 8 | 8 |
| LV-7 | 8 | 8 | 8 | 8 | 8 | 8 |
| LV-8 | 16 | 16 | 8 | 16 | 16 | 8 |
| LV-9 | 8 | 16 | 16 | 16 | 8 | 16 |
| LV-10 | 8 | 8 | 8 | 4 | 8 | 8 |
| LV-11 | 8 | 16 | 8 | 8 | 16 | 8 |
| LV-12 | 8 | 4 | 16 | 8 | 4 | 16 |
| LV-13 | 16 | 4 | 8 | 4 | 8 | 4 |
| LV-14 | 16 | 16 | 4 | 8 | 4 | 8 |
| LV-15 | 8 | 4 | 8 | 8 | 4 | 8 |
| LV-16 | 8 | 4 | 8 | 4 | 16 | 8 |
| LV-17 | 8 | 8 | 16 | 8 | 8 | 8 |
| LV-18 | 4 | 4 | 8 | 2 | 4 | 16 |
| LV-19 | 8 | 16 | 8 | 4 | 8 | 8 |
| LV-20 | 4 | 8 | 8 | 8 | 8 | 8 |
| LV-21 | 8 | 4 | 8 | 8 | 16 | 4 |
| LV-22 | 8 | 8 | 16 | 8 | 8 | 4 |
| LV-23 | 16 | 4 | 8 | 4 | 8 | 16 |
| LV-24 | 8 | 8 | 8 | 8 | 4 | 4 |
| LV-25 | 8 | 8 | 8 | 8 | 8 | 8 |
| LV-26 | 8 | 4 | 16 | 8 | 8 | 16 |
| LV-27 | 8 | 8 | 16 | 4 | 8 | 8 |
| LV-28 | 8 | 4 | 8 | 4 | 8 | 8 |
| LV-29 | 8 | 8 | 4 | 4 | 8 | 16 |
| LV-30 | 8 | 16 | 8 | 8 | 16 | 8 |

### 3. Antimicrobial Experiment of Polypeptides LV-1 to LV-30 Against 5 Clinically Common Drug-Resistant Bacterial Strains

According to the Standards of Clinical and Laboratory Standards Institute (CLSI) of the USA for antimicrobial drug susceptibility test operation standards, the micronutrient serial twofold dilution method was used further determine MICs of the antimicrobial peptides LV-1 to LV-30 against 5 clinically common drug-resistant bacterial strains. The experiment was repeated 5 times in parallel. Final concentrations of polypeptide solutions were 0.125, 0.25, 0.5, 1, 2, 4, 8, 16, 32, 64, and 128 μg/mL, and a lowest drug concentration that completely inhibited the bacterial growth in the wells was identified as the MIC of the AMPs. The tested drug-resistant strains included Methicillin-resistant Staphylococcus aureus (MRSA), carbapenem-resistant Escherichia coli (CREC), vancomycin-resistant Enterococcus (VRE), carbapenem-resistant Pseudomonas aeruginosa (CRPA), and carbapenem-resistant Klebsiella pneumoniae (CRKP). Results are shown in Table 5.

As shown in Table 5, the antimicrobial peptides LV-1 to LV-30 exhibited good antimicrobial activity against all the 5 tested drug-resistant bacterial strains (MIC≥8 μg/mL). The antimicrobial experimental results in Tables 2, 3, 4, and 5 indicated that that the antimicrobial peptides LV-1 to LV-30 of the present disclosure exhibited high-efficiency and broad-spectrum antimicrobial activity, and could kill various microorganisms, including clinically common bacteria, fungi, and drug-resistant strains.

TABLE 5
Experiment results of MICs (μg/mL) of LV-1 to LV-30
against clinically common drug-resistant strains

| Strain type | MRSA | CREC | VRE | CRPA | CRKP |
|---|---|---|---|---|---|
| LV-1 | 4 | 2 | 4 | 4 | 2 |
| LV-2 | 2 | 4 | 2 | 8 | 2 |
| LV-3 | 2 | 4 | 8 | 8 | 4 |
| LV-4 | 8 | 8 | 2 | 4 | 2 |
| LV-5 | 4 | 8 | 8 | 4 | 4 |
| LV-6 | 4 | 2 | 2 | 8 | 8 |
| LV-7 | 2 | 8 | 4 | 2 | 4 |
| LV-8 | 4 | 4 | 8 | 8 | 2 |
| LV-9 | 2 | 2 | 4 | 4 | 8 |
| LV-10 | 4 | 4 | 8 | 2 | 2 |
| LV-11 | 2 | 2 | 4 | 4 | 8 |
| LV-12 | 8 | 8 | 4 | 2 | 4 |
| LV-13 | 2 | 4 | 4 | 2 | 2 |
| LV-14 | 4 | 8 | 4 | 8 | 4 |
| LV-15 | 4 | 4 | 8 | 8 | 8 |
| LV-16 | 4 | 8 | 2 | 8 | 2 |
| LV-17 | 8 | 4 | 8 | 4 | 4 |
| LV-18 | 4 | 2 | 8 | 4 | 2 |
| LV-19 | 4 | 8 | 4 | 2 | 4 |
| LV-20 | 2 | 8 | 4 | 4 | 8 |
| LV-21 | 4 | 2 | 2 | 4 | 2 |
| LV-22 | 2 | 8 | 4 | 8 | 2 |
| LV-23 | 4 | 2 | 4 | 4 | 8 |
| LV-24 | 4 | 8 | 2 | 8 | 4 |
| LV-25 | 2 | 4 | 8 | 4 | 8 |
| LV-26 | 4 | 8 | 2 | 4 | 2 |
| LV-27 | 8 | 4 | 8 | 4 | 4 |
| LV-28 | 4 | 4 | 4 | 8 | 4 |
| LV-29 | 4 | 8 | 4 | 4 | 8 |
| LV-30 | 8 | 4 | 4 | 8 | 4 |

### Example 3 Hemolytic Activity Assay of AMPs

Fresh rabbit blood (500 g) was collected and centrifuged at 500 g for 5 min, a supernatant was discarded, and red blood cells were repeatedly washed with phosphate-buffered saline (PBS, 0.01 M, pH 7.4) until the supernatant was clear. The red blood cells (RBCs) were resuspended to 10 mL, and diluted 25-fold with PBS (0.01 M, pH 7.4) to prepare a red blood cell suspension with a concentration of about 2×10⁸ RBC/mL. The AMPs were subjected to gradient dilution with normal saline (0.85%, w/v, NaCl) according to a multiple dilution method, final concentrations of the SMPs were 1.95, 3.9, 7.8, 15.6, 31.3, 62.5, 125, 250, 500, 1000, and 2000 μg/mL. 100 μl of polypeptide solution was taken and added to a 96-well plate with 5 replicates for each concentration; 100 μl of rabbit blood with a red blood cell count of about 2×10⁸ RBC/mL was then added to each well, the plate was incubated at 37° C. for 1 h, centrifuged at 500 g for 5 min, 100 μl of supernatant was taken and transferred to a new 96-well plate, and an optical density (OD) at a wavelength of 541 nm was measured using a microplate reader. Triton X-100 (10 mg/mL as a starting concentration, two-fold gradient dilution) was used as a positive control group. A hemolysis rate was calculated using the following formula: Hemolysis rate (%)=[(OD541 of experimental group-OD541 of negative control group)/(OD541 of positive control group−OD541 of negative control group)]×100% Specific results of the hemolysis test are shown in Table 6. Minimum hemolytic concentration (MHC): a lowest drug concentration that can cause 10% hemolysis.

As shown in Table 6, when the concentration was far higher than the MIC, all the tested AMPs (LV-1 to LV-60) showed no obvious hemolysis on the rabbit RBCs, indicating that they exhibited good safety.

TABLE 6
MHC of polypeptides for rabbit red blood cells

| S/N | MHC (μg/mL) |
|---|---|
| LV-1 | >1000 |
| LV-2 | >1000 |
| LV-3 | >1000 |
| LV-4 | >1000 |
| LV-5 | >1000 |
| LV-6 | >1000 |
| LV-7 | >1000 |
| LV-8 | >1000 |
| LV-9 | >1000 |
| LV-10 | >1000 |
| LV-11 | >1000 |
| LV-12 | >1000 |
| LV-13 | >1000 |
| LV-14 | >1000 |
| LV-15 | >1000 |
| LV-16 | >1000 |
| LV-17 | >1000 |
| LV-18 | >1000 |
| LV-19 | >1000 |
| LV-20 | >1000 |
| LV-21 | >1000 |
| LV-22 | >1000 |
| LV-23 | >1000 |
| LV-24 | >1000 |
| LV-25 | >1000 |
| LV-26 | >1000 |
| LV-27 | >1000 |
| LV-28 | >1000 |
| LV-29 | >1000 |
| LV-30 | >1000 |
| LV-31 | >1000 |
| LV-32 | >1000 |
| LV-33 | >1000 |
| LV-34 | >1000 |
| LV-35 | >1000 |
| LV-36 | >1000 |
| LV-37 | >1000 |
| LV-38 | >1000 |
| LV-39 | >1000 |
| LV-40 | >1000 |
| LV-41 | >1000 |
| LV-42 | >1000 |
| LV-43 | >1000 |
| LV-44 | >1000 |
| LV-45 | >1000 |
| LV-46 | >1000 |
| LV-47 | >1000 |
| LV-48 | >1000 |
| LV-49 | >1000 |
| LV-50 | >1000 |
| LV-51 | >1000 |
| LV-52 | >1000 |
| LV-53 | >1000 |
| LV-54 | >1000 |
| LV-55 | >1000 |
| LV-56 | >1000 |
| LV-57 | >1000 |
| LV-58 | >1000 |
| LV-59 | >1000 |
| LV-60 | >1000 |
| Triton X-100 | 156 |

### Example 4 Cytotoxicity Assay of AMPs

HEK293T cells (sourced from the American Type Culture Collection) were cultured in A DMEM medium containing 10% fetal bovine serum, and incubated in a 37° C. incubator with 5% CO₂. Cells in a logarithmic phase were collected, adherent cells were treated with 0.25% trypsin, and a concentration of cell suspension was adjusted to 5×10⁴ cells/mL; 100 μL of the cell suspension was added to each well of a 96-well plate, the 96-well plate was placed in a cell culture incubator, and incubated at 37° C. with 5% CO₂ for 24 h, and the culture medium was removed; the cells were washed twice with PBS, 100 μL of AMP solution (with a final concentration of 128 μg/mL) was added to each well; 5 replicates were set for each concentration, the 96-well plate was continuously incubated in the cell culture incubator at 37° C. with 5% CO₂ for another 48 h; 20 μL of MTT solution (5 mg/mL) was added to each well and incubated for another 4 h; the culture medium in the 96-well plate was carefully removed, and 150 μL of DMSO solvent was added to each well, the plate was generally shaken to dissolve crystal therein, and an absorbance at a wavelength of 570 nm (OD value) was measured using the microplate reader. A cell viability was calculated using the following formula: Cell viability (%)=(OD value of experiment group−OD value of blank control group)/(OD value of control group−OD value of blank control group)×100%.

Cytotoxicity experimental results are shown in FIGURE. The AMPs LV-1 to LV-30 described in the present disclosure had no significant effect on the viability of HEK293T cells at a high concentration of 128 μg/mL, indicating that the AMPs have no obvious toxic effects on mammalian immune cells, and can effectively distinguish bacterial cells from mammalian cells, demonstrating high safety. Therefore, the AMPs have promising application prospects in the fields of preparing antimicrobial drugs, antimicrobial formulations, and other antimicrobial products.

### Example 5 Protease Stability Assay of AMPs

Trypsin (250 U/mg, pH=8.0) and AMPs (LV-1 to LV-30) were mixed in a ratio of 1:10 (w/w) and added to a reaction buffer (0.2 M sodium phosphate, 1 mM CaCl₂), pH 7.5), and then incubated at 37° C. for 3 h. According to the experimental method described in Example 2, polypeptide solution and Staphylococcus aureus (NRS 384) bacterial suspension were taken and added to a sterile 96-well cell culture plate, 5 replicates were set for each concentration; concentrations of the antimicrobial peptide were 0.125, 0.25, 0.5, 1, 2, 4, 8, 16, 32, 64, to 128 μg/mL; the plate was incubated in a constant-temperature incubator at 37° C. for 16-24 h until visible turbidity appeared in the negative control wells, and MIC values of the AMPs after trypsin treatment were observed.

Experimental results are shown in Table 7. After the AMPs LV-1 to LV-30 described in the present disclosure were treated with trypsin, their MIC values remained unchanged. In contrast, the positive control L-type AMP Cbf-K₁₆ lost its antimicrobial activity after being treated with trypsin, indicating that peptide bonds formed by D-amino acids have stronger enzyme resistance than that of L-amino acids, which significantly improves the clinical application value of the AMPs.

TABLE 7
Protease stability assay of AMPs

| | Trypsin MIC |
|---|---|
| S/N | (μg/mL) |
| LV-1 (All D-type) | 1 |
| LV-2 (All D-type) | 1 |
| LV-3 (All D-type) | 1 |
| LV-4 (All D-type) | 1 |
| LV-5 (All D-type) | 1 |
| LV-6 (All D-type) | 1 |
| LV-7 (All D-type) | 1 |
| LV-8 (All D-type) | 1 |
| LV-9 (All D-type) | 1 |
| LV-10 (All D-type) | 1 |
| LV-11 (All D-type) | 1 |
| LV-12 (All D-type) | 1 |
| LV-13 (All D-type) | 1 |
| LV-14 (All D-type) | 1 |
| LV-15 (All D-type) | 1 |
| LV-16 (All D-type) | 1 |
| LV-17 (All D-type) | 1 |
| LV-18 (All D-type) | 1 |
| LV-19 (All D-type) | 1 |
| LV-20 (All D-type) | 1 |
| LV-21 (All D-type) | 1 |
| LV-22 (All D-type) | 1 |
| LV-23 (All D-type) | 1 |
| LV-24 (All D-type) | 1 |
| LV-25 (All D-type) | 1 |
| LV-26 (All D-type) | 1 |
| LV-27 (All D-type) | 1 |
| LV-28 (All D-type) | 1 |
| LV-29 (All D-type) | 1 |
| LV-30 (All D-type) | 1 |
| Cbf-K₁₆ | >128 |

### Example 6 Serum Stability Assay of AMPs LV-1 to LV-30

30 μL of each AMP (LV-1 to LV-30) solution (10 mg/mL stock solution) was added to 300 μL of human serum for incubation, and incubated at 37° C. for 12 h, samples were then 5 collected, a residual level of AMPs in human serum was measured using reverse-phase high-performance liquid chromatography.

Results are shown in Table 8. After incubation of the AMPs LV-1 to LV-30 in human serum for 12 h, the residual level of AMPs remained above 97%, indicating that the AMPs LV-1 to LV-30 exhibited good stability in human serum.

TABLE 8
Residual level of AMPs in human serum

| S/N | Residual level (%) |
|---|---|
| LV-1 | 98.45% |
| LV-2 | 97.36% |
| LV-3 | 98.68% |
| LV-4 | 98.72% |
| LV-5 | 98.15% |
| LV-6 | 98.03% |
| LV-7 | 98.86% |
| LV-8 | 97.61% |
| LV-9 | 97.29% |
| LV-10 | 98.01% |
| LV-11 | 98.35% |
| LV-12 | 97.48% |
| LV-13 | 98.55% |
| LV-14 | 97.09% |
| LV-15 | 97.32% |
| LV-16 | 98.37% |
| LV-17 | 97.17% |
| LV-18 | 97.64% |
| LV-19 | 98.11% |
| LV-20 | 98.24% |
| LV-21 | 97.93% |
| LV-22 | 97.19% |
| LV-23 | 97.52% |
| LV-24 | 98.27% |
| LV-25 | 97.75% |
| LV-26 | 97.82% |
| LV-27 | 98.75% |
| LV-28 | 98.18% |
| LV-29 | 98.36% |
| LV-30 | 98.07% |

### Example 7 Therapeutic Study of Topical Formulations of AMPs LV-1 to LV-30 on Back Skin Infections in Mice

Both female and male ICR mice were used in the test, half of the female and male mice were taken and randomly assigned into groups, with 10 mice in each group, and kept in separate cages. The mice were divided into a blank group, a model group, a positive control Methicillin group, and topical formulation groups of AMPs LV-1 to LV-30. Suspensions of Methicillin-sensitive Staphylococcus aureus (NRS 384) and Methicillin-resistant Staphylococcus aureus (MRSA) were prepared, and their concentrations were adjusted to a concentration of 5×10⁶ CFU/mL for subsequent use. Hair on backs of the mice was shaved with a shaver, wound was created on back skin of each mouse using a biopsy punch, and the wound was then infected with Methicillin-sensitive Staphylococcus aureus and Methicillin-resistant Staphylococcus aureus (0.1 mL, 5×10⁶ CFU/mL) to establish infection models. Except for the blank group and the model group, all other groups received corresponding topical formulations applied directly to the infected areas, with a dose of 0.1 mL, twice a day for 4 consecutive days. On the day following the final administration, skin from the infected sites of each group was aseptically collected, and an antibacterial rate was calculated. Results are shown in Table 9.

As shown in Table 9, the groups with high, medium, and low doses of AMPs LV-1 to LV-30 not only had an inhibition rate of more than 95% against Methicillin-sensitive Staphylococcus aureus, but also had an inhibition rate of more than 85% against Methicillin-resistant Staphylococcus aureus, which is significantly better than the Methicillin group, demonstrating that the topical formulations of AMPs LV-1 to LV-30 exhibited significant inhibitory effect on skin wound infected with Staphylococcus aureus.

TABLE 9
Effect of topical formulations of AMPs LV-1
to LV-30 on skin infections (n = 10)

| | | Inhibition | Inhibition |
|---|---|---|---|
| | | rate against | rate against |
| | | Methicillin- | Methicillin- |
| | Number of | sensitive | resistant |
| | animal | Staphylococcus | Staphylococcus |
| Group | (Mouse) | aureus (%) | aureus (%) |
| Blank group | 10 | 0 | 0 |
| Model group | 10 | — | — |
| Methicillin | 10 | 84.83 | 1.05 |
| group (1 mg/mL) | | | |
| LV-1 (0.2 mg/mL) | 10 | 96.77 | 86.32 |
| LV-1 (1 mg/mL) | 10 | 98.64 | 89.47 |
| LV-1 (5 mg/mL) | 10 | 97.49 | 87.81 |
| LV-2 (0.2 mg/mL) | 10 | 97.51 | 87.14 |
| LV-2 (1 mg/mL) | 10 | 99.02 | 90.35 |
| LV-2 (5 mg/mL) | 10 | 98.49 | 88.56 |
| LV-3 (0.2 mg/mL) | 10 | 96.08 | 86.42 |
| LV-3 (1 mg/mL) | 10 | 97.43 | 87.69 |
| LV-3 (5 mg/mL) | 10 | 98.11 | 87.93 |
| LV-4 (0.2 mg/mL) | 10 | 96.05 | 86.18 |
| LV-4 (1 mg/mL) | 10 | 96.76 | 86.52 |
| LV-4 (5 mg/mL) | 10 | 97.44 | 87.39 |
| LV-5 (0.2 mg/mL) | 10 | 96.37 | 86.17 |
| LV-5 (1 mg/mL) | 10 | 97.01 | 86.66 |
| LV-5 (5 mg/mL) | 10 | 97.75 | 87.40 |
| LV-6 (0.2 mg/mL) | 10 | 97.16 | 86.72 |
| LV-6 (1 mg/mL) | 10 | 98.78 | 89.80 |
| LV-6 (5 mg/mL) | 10 | 97.51 | 88.14 |
| LV-7 (0.2 mg/mL) | 10 | 96.48 | 87.61 |
| LV-7 (1 mg/mL) | 10 | 97.94 | 88.45 |
| LV-7 (5 mg/mL) | 10 | 97.39 | 88.12 |
| LV-8 (0.2 mg/mL) | 10 | 96.27 | 86.26 |
| LV-8 (1 mg/mL) | 10 | 97.13 | 86.92 |
| LV-8 (5 mg/mL) | 10 | 98.29 | 87.24 |
| LV-9 (0.2 mg/mL) | 10 | 96.38 | 86.16 |
| LV-9 (1 mg/mL) | 10 | 96.95 | 86.82 |
| LV-9 (5 mg/mL) | 10 | 97.67 | 87.81 |
| LV-10 (0.2 mg/mL) | 10 | 96.31 | 87.04 |
| LV-10 (1 mg/mL) | 10 | 97.46 | 87.83 |
| LV-10 (5 mg/mL) | 10 | 98.97 | 88.09 |
| LV-11 (0.2 mg/mL) | 10 | 96.29 | 86.12 |
| LV-11 (1 mg/mL) | 10 | 97.21 | 86.35 |
| LV-11 (5 mg/mL) | 10 | 98.08 | 87.64 |
| LV-12 (0.2 mg/mL) | 10 | 96.94 | 86.43 |
| LV-12 (1 mg/mL) | 10 | 98.69 | 89.61 |
| LV-12 (5 mg/mL) | 10 | 97.53 | 87.97 |
| LV-13 (0.2 mg/mL) | 10 | 96.06 | 86.05 |
| LV-13 (1 mg/mL) | 10 | 96.15 | 86.22 |
| LV-13 (5 mg/mL) | 10 | 97.28 | 87.97 |
| LV-14 (0.2 mg/mL) | 10 | 96.51 | 86.13 |
| LV-14 (1 mg/mL) | 10 | 97.04 | 86.65 |
| LV-14 (5 mg/mL) | 10 | 98.83 | 87.28 |
| LV-15 (0.2 mg/mL) | 10 | 96.22 | 86.01 |
| LV-15 (1 mg/mL) | 10 | 96.69 | 86.42 |
| LV-15 (5 mg/mL) | 10 | 97.56 | 87.81 |
| LV-16 (0.2 mg/mL) | 10 | 96.24 | 86.05 |
| LV-16 (1 mg/mL) | 10 | 96.91 | 86.76 |
| LV-16 (5 mg/mL) | 10 | 98.18 | 87.84 |
| LV-17 (0.2 mg/mL) | 10 | 97.42 | 87.05 |
| LV-17 (1 mg/mL) | 10 | 98.95 | 89.91 |
| LV-17 (5 mg/mL) | 10 | 97.83 | 88.37 |
| LV-18 (0.2 mg/mL) | 10 | 96.64 | 86.52 |
| LV-18 (1 mg/mL) | 10 | 98.52 | 89.49 |
| LV-18 (5 mg/mL) | 10 | 97.16 | 87.30 |
| LV-19 (0.2 mg/mL) | 10 | 96.35 | 86.28 |
| LV-19 (1 mg/mL) | 10 | 98.19 | 89.33 |
| LV-19 (5 mg/mL) | 10 | 97.04 | 87.16 |
| LV-20 (0.2 mg/mL) | 10 | 97.63 | 87.24 |
| LV-20 (1 mg/mL) | 10 | 99.14 | 90.05 |
| LV-20 (5 mg/mL) | 10 | 98.62 | 88.71 |
| LV-21 (0.2 mg/mL) | 10 | 96.49 | 86.34 |
| LV-21 (1 mg/mL) | 10 | 97.38 | 86.72 |
| LV-21 (5 mg/mL) | 10 | 98.25 | 87.69 |
| LV-22 (0.2 mg/mL) | 10 | 96.20 | 86.11 |
| LV-22 (1 mg/mL) | 10 | 96.83 | 87.34 |
| LV-22 (5 mg/mL) | 10 | 97.77 | 88.05 |
| LV-23 (0.2 mg/mL) | 10 | 96.12 | 86.64 |
| LV-23 (1 mg/mL) | 10 | 97.44 | 87.29 |
| LV-23 (5 mg/mL) | 10 | 98.86 | 88.63 |
| LV-24 (0.2 mg/mL) | 10 | 96.25 | 86.47 |
| LV-24 (1 mg/mL) | 10 | 97.03 | 87.15 |
| LV-24 (5 mg/mL) | 10 | 98.91 | 88.72 |
| LV-25 (0.2 mg/mL) | 10 | 97.25 | 86.94 |
| LV-25 (1 mg/mL) | 10 | 98.87 | 89.83 |
| LV-25 (5 mg/mL) | 10 | 97.76 | 88.24 |
| LV-26 (0.2 mg/mL) | 10 | 96.35 | 86.51 |
| LV-26 (1 mg/mL) | 10 | 96.92 | 86.79 |
| LV-26 (5 mg/mL) | 10 | 97.87 | 87.56 |
| LV-27 (0.2 mg/mL) | 10 | 97.01 | 86.50 |
| LV-27 (1 mg/mL) | 10 | 98.74 | 89.66 |
| LV-27 (5 mg/mL) | 10 | 97.65 | 88.02 |
| LV-28 (0.2 mg/mL) | 10 | 96.03 | 86.23 |
| LV-28 (1 mg/mL) | 10 | 96.75 | 87.81 |
| LV-28 (5 mg/mL) | 10 | 97.82 | 88.66 |
| LV-29 (0.2 mg/mL) | 10 | 96.49 | 86.34 |
| LV-29 (1 mg/mL) | 10 | 97.11 | 87.29 |
| LV-29 (5 mg/mL) | 10 | 98.65 | 88.16 |
| LV-30 (0.2 mg/mL) | 10 | 96.07 | 86.10 |
| LV-30 (1 mg/mL) | 10 | 96.91 | 87.85 |
| LV-30 (5 mg/mL) | 10 | 98.24 | 88.93 |

### Example 8 Therapeutic Study of Injectable Formulations of AMPs LV-1 to LV-30 on Mice Infected with Peritonitis

Both female and male ICR mice were used in the test, half of the female and male mice were taken and randomly assigned into groups, with 10 mice in each group, and kept in separate cages. The mice were divided into a blank control group, a negative control group, a positive control Levofloxacin group, and AMPs LV-1 to LV-30 groups. A bacterial suspension of Pseudomonas aeruginosa ATCC 27853 was prepared, a concentration of the suspension was adjusted 1×10⁹ CFU/mL for subsequent use. Except for the blank control group, all other mice were injected with the bacterial suspension (0.2 mL, 1×10⁹ CFU/mL), into abdominal cavity of the mice, to establish a model. After the model was successfully established, the mice were given drug immediately according to their body weights. Except for the blank control group and the negative control group, the mice in all other groups were administered the corresponding injectable formulations via tail vein, twice a day for 3 consecutive days. The mice were then observed for 14 d. The death of mice in each group was observed, and survival time of the mice was recorded. Results of the effect of the injectable formulations of AMPs LV-1 to LV-30 on the survival time of mice infected with Pseudomonas aeruginosa are shown in Table 10.

As shown in Table 10, all 10 mice in the negative control group died within 2 days (10/10) after modeling. There was a greatly significant difference in an average survival day of the mice between the AMPs LV-1 to LV-30 groups and the negative control group (**P<0.01, ***P<0.001, or ****P<0.0001), indicating that the AMPs LV-1 to LV-30 had a good anti-infective effect. Although the positive control Levofloxacin group could effectively prolong the survival time of the infected mice, the survival time of infected mice in the AMPs LV-1˜LV-30 groups was significantly longer than that in the positive control Levofloxacin group. Therefore, it can be seen that the injectable formulations of AMPs LV-1 to LV-30 had a good in vivo therapeutic effect on the mice infected with Pseudomonas aeruginosa. In addition, from all 30 AMPs (LV-1 to LV-30) with a common motif structure, 10 representative polypeptides (LV-1, LV-2, LV-6, LV-12, LV-17, LV-18, LV-19, LV-20, LV-25, and LV-27) were selected for efficacy experiment with high, medium, and low doses in mice infected with peritonitis. As shown in Table 10, there were extremely significant differences in average survival days of mice in the high, medium and low dose groups of the 10 polypeptides compared with the negative control group (**P<0.01, ***P<0.001 or ****P<0.0001), and the therapeutic effects in the high, medium and low dose groups were superior to those of the Levofloxacin group.

TABLE 10
Effect of the injectable formulations of AMPs
LV-1 to LV-30 on the survival time of mice infected
with Pseudomonas aeruginosa (n = 10)

| | | Number of mice that died on | | | | | Average |
|---|---|---|---|---|---|---|---|
| | Dose | each day after infection (Mouse) | | | | | survival |
| Group | (mg/kg) | 1 | 2 | 3 | 4 | 5 | times (Days) |
| Blank | — | 0 | 0 | 0 | 0 | 0 | — |
| control | | | | | | | |
| Negative | — | 7 | 3 | 0 | 0 | 0 | 1.3 |
| control | | | | | | | |
| Levofloxacin | 5 | 3 | 1 | 1 | 1 | 1 | 5.9* |
| LV-1 | 0.2 | 1 | 2 | 1 | 1 | 1 | 7.3** |
| LV-1 | 1 | 0 | 1 | 1 | 1 | 0 | 10.7**** |
| LV-1 | 5 | 2 | 1 | 1 | 0 | 0 | 9.1** |
| LV-2 | 0.2 | 3 | 1 | 1 | 0 | 0 | 7.8** |
| LV-2 | 1 | 0 | 0 | 1 | 0 | 1 | 12.0**** |
| LV-2 | 5 | 1 | 0 | 1 | 0 | 0 | 11.6**** |
| LV-3 | 1 | 1 | 1 | 0 | 2 | 1 | 8.6** |
| LV-4 | 1 | 0 | 1 | 0 | 2 | 1 | 9.9**** |
| LV-5 | 1 | 1 | 1 | 1 | 0 | 1 | 9.5*** |
| LV-6 | 0.2 | 2 | 2 | 1 | 0 | 0 | 7.9** |
| LV-6 | 1 | 1 | 1 | 0 | 1 | 0 | 10.5**** |
| LV-6 | 5 | 1 | 2 | 1 | 0 | 0 | 9.2*** |
| LV-7 | 1 | 0 | 1 | 1 | 1 | 1 | 9.8*** |
| LV-8 | 1 | 1 | 1 | 0 | 1 | 1 | 9.6*** |
| LV-9 | 1 | 1 | 2 | 0 | 1 | 0 | 9.3*** |
| LV-10 | 1 | 0 | 1 | 1 | 2 | 0 | 9.7*** |
| LV-11 | 1 | 0 | 1 | 1 | 0 | 2 | 9.9**** |
| LV-12 | 0.2 | 2 | 1 | 0 | 2 | 1 | 7.3** |
| LV-12 | 1 | 0 | 0 | 2 | 0 | 1 | 10.9**** |
| LV-12 | 5 | 0 | 1 | 2 | 0 | 1 | 9.7*** |
| LV-13 | 1 | 3 | 1 | 0 | 1 | 0 | 7.9** |
| LV-14 | 1 | 1 | 2 | 0 | 1 | 1 | 8.4** |
| LV-15 | 1 | 1 | 2 | 0 | 2 | 0 | 8.3** |
| LV-16 | 1 | 2 | 1 | 1 | 1 | 0 | 8.1** |
| LV-17 | 0.2 | 1 | 2 | 1 | 1 | 1 | 7.3** |
| LV-17 | 1 | 0 | 2 | 0 | 1 | 0 | 10.6**** |
| LV-17 | 5 | 0 | 2 | 1 | 1 | 0 | 9.5*** |
| LV-18 | 0.2 | 2 | 1 | 1 | 1 | 1 | 7.2** |
| LV-18 | 1 | 0 | 1 | 0 | 1 | 1 | 10.9**** |
| LV-18 | 5 | 0 | 2 | 1 | 0 | 0 | 10.5**** |
| LV-19 | 0.2 | 3 | 2 | 0 | 0 | 0 | 7.7** |
| LV-19 | 1 | 0 | 0 | 1 | 0 | 2 | 11.1**** |
| LV-19 | 5 | 1 | 0 | 2 | 0 | 0 | 10.5**** |
| LV-20 | 0.2 | 2 | 1 | 2 | 1 | 0 | 7.0** |
| LV-20 | 1 | 1 | 1 | 1 | 0 | 0 | 10.4**** |
| LV-20 | 5 | 1 | 2 | 0 | 0 | 0 | 10.3**** |
| LV-21 | 1 | 2 | 0 | 2 | 0 | 0 | 9.2*** |
| LV-22 | 1 | 1 | 1 | 0 | 1 | 1 | 9.6*** |
| LV-23 | 1 | 1 | 1 | 0 | 2 | 0 | 9.5*** |
| LV-24 | 1 | 1 | 2 | 0 | 0 | 1 | 9.4*** |
| LV-25 | 0.2 | 2 | 1 | 1 | 1 | 1 | 7.2** |
| LV-25 | 1 | 0 | 1 | 1 | 0 | 1 | 10.8**** |
| LV-25 | 5 | 0 | 3 | 0 | 0 | 0 | 10.4**** |
| LV-26 | 1 | 1 | 1 | 1 | 1 | 1 | 8.5** |
| LV-27 | 0.2 | 2 | 2 | 0 | 1 | 1 | 7.1** |
| LV-27 | 1 | 1 | 1 | 0 | 1 | 0 | 10.5**** |
| LV-27 | 5 | 0 | 3 | 0 | 1 | 0 | 9.4*** |
| LV-28 | 1 | 1 | 0 | 1 | 1 | 1 | 9.7*** |
| LV-29 | 1 | 1 | 0 | 0 | 3 | 1 | 8.8** |
| LV-30 | 1 | 1 | 1 | 0 | 1 | 1 | 9.6*** |

Note:
Compared with the negative control group *P < 0.05, **P < 0.01, ***P < 0.001, ****P < 0.0001

### Example 9 Therapeutic Study of Oral Formulations of AMPs LV-1 to LV-30 on Mice with Systemic Bacterial Infection

Both female and male ICR mice were used in the test, half of the female and male mice were taken and randomly assigned into groups, with 10 mice in each group, and kept in separate cages. The mice were divided into a blank control group, a negative control group, a positive control Levofloxacin group, and AMPs LV-1 to LV-30 groups. An Escherichia coli ATCC 25922 bacterial suspension was prepared, a concentration of the suspension was adjusted 1×10⁷ CFU/mL for subsequent use. Except for the blank control group, all other mice were injected with the Escherichia coli suspension (0.2 mL, 1×10⁷ CFU/mL), into abdominal cavity of the mice, to establish a model. After the model was successfully established, the mice were given drug immediately according to their body weights. Except for the blank control group and the negative control group, the mice in all other groups were gavaged with different compounds, three times a day for 7 consecutive days. The mice were then observed for 14 d. The death of mice in each group was observed, and survival time of the mice was recorded. Results of the effect of the oral formulations of AMPs LV-1 to LV-30 on the survival time of mice infected with Escherichia coli are shown in Table 11.

As shown in Table 11, all 10 mice in the negative control group died within 5 days (10/10) after modeling. There was a significant difference in an average survival day of the mice between the AMPs LV-1 to LV-30 groups and the negative control group (**P<0.01, ***P<0.001, or ****P<0.0001), indicating that the AMPs LV-1 to LV-30 had a good anti-infective effect. Although the positive control Levofloxacin group could effectively prolong the survival time of the infected mice, the survival time of infected mice in the AMPs LV-1˜LV-30 groups was significantly longer than that in the positive control Levofloxacin group. Therefore, it can be seen that the oral formulations of AMPs LV-1 to LV-30 had a good in vivo therapeutic effect on the mice infected with Escherichia coli. In addition, from all 30 AMPs (LV-1 to LV-30) with a common motif structure, 10 representative polypeptides (LV-1, LV-2, LV-6, LV-12, LV-17, LV-18, LV-19, LV-20, LV-25, and LV-27) were selected for efficacy experiment with high, medium, and low doses in mice with systemic bacterial infection. As shown in Table 11, there were extremely significant differences in average survival days of mice in the high, medium and low dose groups of the 10 polypeptides compared with the negative control group (**P<0.01, ***P<0.001 or ****P<0.0001), and the therapeutic effects in the high, medium and low dose groups were superior to those of the Levofloxacin group.

TABLE 11
Effect of the oral formulations of AMPs LV-1 to LV-30 on the survival
time of mice infected with Escherichia coli (n = 10)

| | | Number of mice that died on | | | | | Average |
|---|---|---|---|---|---|---|---|
| | Dose | each day after infection (Mouse) | | | | | survival |
| Group | (mg/kg) | 4 | 5 | 6 | 7 | 8 | times (Days) |
| Blank | — | 0 | 0 | 0 | 0 | 0 | — |
| control | | | | | | | |
| Negative | — | 6 | 4 | 0 | 0 | 0 | 4.4 |
| control | | | | | | | |
| Levofloxacin | 5 | 2 | 2 | 1 | 1 | 1 | 8.1* |
| LV-1 | 0.2 | 0 | 3 | 1 | 0 | 1 | 9.9** |
| LV-1 | 1 | 0 | 0 | 1 | 1 | 1 | 11.9**** |
| LV-1 | 5 | 0 | 0 | 2 | 1 | 0 | 11.7**** |
| LV-2 | 0.2 | 1 | 2 | 1 | 1 | 1 | 9.1** |
| LV-2 | 1 | 0 | 2 | 1 | 0 | 0 | 11.4**** |
| LV-2 | 5 | 0 | 3 | 1 | 0 | 0 | 10.5*** |
| LV-3 | 1 | 1 | 0 | 1 | 2 | 1 | 10.2*** |
| LV-4 | 1 | 0 | 1 | 2 | 2 | 1 | 9.5** |
| LV-5 | 1 | 1 | 2 | 2 | 1 | 0 | 8.9** |
| LV-6 | 0.2 | 1 | 3 | 2 | 0 | 0 | 8.7** |
| LV-6 | 1 | 1 | 0 | 1 | 1 | 1 | 10.9*** |
| LV-6 | 5 | 2 | 1 | 1 | 0 | 0 | 10.3*** |
| LV-7 | 1 | 2 | 2 | 0 | 1 | 1 | 8.9** |
| LV-8 | 1 | 2 | 1 | 0 | 2 | 0 | 9.7** |
| LV-9 | 1 | 1 | 1 | 1 | 2 | 0 | 9.9** |
| LV-10 | 1 | 0 | 2 | 2 | 0 | 1 | 10.0*** |
| LV-11 | 1 | 1 | 1 | 1 | 1 | 2 | 9.4** |
| LV-12 | 0.2 | 1 | 2 | 2 | 1 | 0 | 8.9** |
| LV-12 | 1 | 0 | 1 | 0 | 2 | 1 | 11.1**** |
| LV-12 | 5 | 0 | 2 | 1 | 1 | 0 | 10.7*** |
| LV-13 | 1 | 3 | 1 | 1 | 0 | 0 | 9.3** |
| LV-14 | 1 | 1 | 2 | 1 | 0 | 1 | 9.8** |
| LV-15 | 1 | 1 | 3 | 0 | 1 | 0 | 9.6** |
| LV-16 | 1 | 1 | 1 | 1 | 0 | 2 | 10.1*** |
| LV-17 | 0.2 | 1 | 2 | 1 | 1 | 2 | 8.5** |
| LV-17 | 1 | 1 | 0 | 2 | 1 | 0 | 10.7*** |
| LV-17 | 5 | 2 | 1 | 0 | 1 | 1 | 9.8** |
| LV-18 | 0.2 | 0 | 1 | 2 | 1 | 0 | 10.8*** |
| LV-18 | 1 | 0 | 1 | 0 | 1 | 0 | 12.4**** |
| LV-18 | 5 | 0 | 1 | 2 | 0 | 0 | 11.5**** |
| LV-19 | 0.2 | 0 | 2 | 1 | 2 | 1 | 9.4** |
| LV-19 | 1 | 0 | 1 | 1 | 1 | 0 | 11.6**** |
| LV-19 | 5 | 0 | 2 | 1 | 1 | 0 | 10.7*** |
| LV-20 | 0.2 | 1 | 1 | 2 | 2 | 0 | 9.1** |
| LV-20 | 1 | 1 | 1 | 0 | 1 | 0 | 11.4**** |
| LV-20 | 5 | 2 | 0 | 2 | 0 | 0 | 10.4*** |
| LV-21 | 1 | 1 | 2 | 1 | 0 | 1 | 9.8** |
| LV-22 | 1 | 0 | 2 | 1 | 1 | 2 | 9.5** |
| LV-23 | 1 | 2 | 1 | 1 | 0 | 1 | 9.7** |
| LV-24 | 1 | 1 | 1 | 0 | 2 | 1 | 10.1*** |
| LV-25 | 0.2 | 1 | 1 | 2 | 2 | 1 | 8.5** |
| LV-25 | 1 | 1 | 1 | 0 | 1 | 1 | 10.8*** |
| LV-25 | 5 | 0 | 3 | 0 | 2 | 0 | 9.9** |
| LV-26 | 1 | 2 | 1 | 0 | 1 | 1 | 9.8** |
| LV-27 | 0.2 | 1 | 1 | 2 | 1 | 0 | 9.8** |
| LV-27 | 1 | 0 | 1 | 1 | 0 | 1 | 11.7**** |
| LV-27 | 5 | 0 | 2 | 1 | 0 | 0 | 11.4**** |
| LV-28 | 1 | 1 | 2 | 1 | 1 | 0 | 9.7** |
| LV-29 | 1 | 0 | 1 | 2 | 1 | 1 | 10.2*** |
| LV-30 | 1 | 1 | 1 | 3 | 0 | 1 | 9.1** |

Note:
Compared with the negative control group *P < 0.05, **P < 0.01, ***P < 0.001, ****P < 0.0001

It can be seen from the above examples that the AMP described in the present disclosure is a highly effective antimicrobial peptide, which have the characteristics of low molecular weight, broad antibacterial spectrum, high efficacy, non-cytotoxicity, non-hemolysis, and high stability, and a low propensity for inducing drug resistance. In addition, they are capable of killing various microorganisms, including clinically common drug-resistant bacterial strains, bacteria and fungi. Furthermore, they have simple structures, are easily synthesized on a large scale and are cost-effective. Therefore, they are expected to replace conventional antibiotics and become a safe, green, efficient and ideal antimicrobial agent, and have promising application prospects in the preparation of antimicrobial drugs, antimicrobial formulations, and antimicrobial products.

What is claimed is:

1. An antimicrobial peptide, wherein the antimicrobial peptide (AMP) or a pharmaceutical composition thereof has a common amino acid general formula of: Ac-Arg-Leu-Leu-B-Z-Leu-Z-B-NH₂, or Ac-Arg-Leu-Leu-B-Z-Leu-Z-B-Arg-NH₂, or Ac-Arg-Arg-Leu-Leu-B-Z-Leu-Z-B-Arg-Arg-NH₂, wherein Ac represents acetylation modification at an N-terminal of the antimicrobial peptide, NH₂ represents amidation modification at a C-terminal of the antimicrobial peptide, B is selected from aromatic amino acid residues, and one or two Zs are selected from arginine residues or lysine residues.

2. The antimicrobial peptide according to claim 1, wherein the aromatic amino acid residues comprise a 3-(2-naphthyl)-alanine residue, a phenylalanine residue and a phenylalanine-derived residue thereof.

3. The antimicrobial peptide according to claim 1, wherein one or more Arg residues in the common amino acid general formula are substituted with lys residues.

4. The antimicrobial peptide according to claim 1, wherein one or more Leu residues in the common amino acid general formula are substituted with norleucine residues, norvaline residues, homoleucine residues, alanine residues or valine residues.

5. The antimicrobial peptide according to claim 1, wherein the AMP comprises all L-enantiomers or all D-enantiomers; or one or more amino acids in the common amino acid general formula are substituted with L-amino acids or D-amino acids.

6. The antimicrobial peptide according to claim 1, wherein polyethylene glycol (PEG) is conjugated to the N-terminal or the C-terminal of the AMP, or a side-chain amino group of the lys residue.

7. The antimicrobial peptide according to claim 6, wherein a molecular weight of the PEG is 200-4000.

8. The antimicrobial peptide according to claim 1, wherein sequences of the AMP are shown in the following table:

| | Name of | Amino acid sequence | | | | | | | | | | |
|---|---|---|---|---|---|---|---|---|---|---|---|---|
| S/N | AMP | 1 | 2 | 3 | 4 | 5 | 6 | 7 | 8 | 9 | 10 | 11 |
| D-enantiomer | | D | D | D | D | D | D | D | D | D | D | D |
| 1 | LV-1 | R | R | L | L | X | R | L | R | X | R | R |
| 2 | LV-2 | R | K | L | L | F | R | L | K | F | R | K |
| 3 | LV-3 | R | K | L | L | F | R | L | K | X | R | K |
| 4 | LV-4 | R | K | L | L | X | R | L | K | F | R | K |
| 5 | LV-5 | R | K | L | L | X1 | K | L | K | X1 | K | R |
| 6 | LV-6 | R | K | L | L | X2 | K | L | K | X2 | K | R |
| 7 | LV-7 | R | K | L | L | X3 | K | L | K | X3 | K | R |
| 8 | LV-8 | R | K | L | L | X4 | K | L | K | X4 | K | R |
| 9 | LV-9 | R | K | L | L | X5 | K | L | K | X5 | K | R |
| 10 | LV-10 | K | K | L | L | X6 | R | L | K | X6 | R | K |
| 11 | LV-11 | R | K | L | L | X7 | K | L | K | X7 | K | R |
| 12 | LV-12 | R | K | X8 | X8 | X | R | X8 | K | X | R | K |
| 13 | LV-13 | R | K | X9 | X9 | X | R | X9 | K | X | R | K |
| 14 | LV-14 | R | K | X10 | X10 | X | R | X10 | K | X | R | K |
| 15 | LV-15 | R | K | A | A | X | R | A | K | X | R | K |
| 16 | LV-16 | R | K | V | V | X | R | V | K | X | R | K |
| 17 | LV-17 | K | K | L | L | X | K | L | K | X | K | K |
| 18 | LV-18 | R | K | L | L | X | K | L | K | X | K | R |
| 19 | LV-19 | K | K | L | L | X | R | L | K | X | R | K |
| 20 | LV-20 | R | K | L | L | X | R | L | K | X | R | K |
| 21 | LV-21 | R | R | L | L | X | R | L | K | X | R | K |
| 22 | LV-22 | R | K | L | L | X | R | L | K | X | R | R |
| 23 | LV-23 | K | R | L | L | X | K | L | K | X | K | R |
| 24 | LV-24 | R | R | L | L | X | K | L | K | X | K | R |
| 25 | LV-25 | K | K | L | L | X2 | R | L | K | X2 | R | K |
| 26 | LV-26 | Ac-RKLLXRLKXRK-PEG200 | | | | | | | | | | |
| 27 | LV-27 | Ac-RKLLXRLKXRK-PEG500 | | | | | | | | | | |
| 28 | LV-28 | Ac-RKLLXRLKXRK-PEG1000 | | | | | | | | | | |
| 29 | LV-29 | PEG2000-RKLLXRLKXRK-NH₂ | | | | | | | | | | |
| 30 | LV-30 | Ac-RKLLXRLKXRK-PEG4000 | | | | | | | | | | |
| L-enantiomer | | L | L | L | L | L | L | L | L | L | L | L |
| 31 | LV-31 | R | R | L | L | X | R | L | R | X | R | R |
| 32 | LV-32 | R | K | L | L | F | R | L | K | F | R | K |
| 33 | LV-33 | R | K | L | L | F | R | L | K | X | R | K |
| 34 | LV-34 | R | K | L | L | X | R | L | K | F | R | K |
| 35 | LV-35 | R | K | L | L | X1 | K | L | K | X1 | K | R |
| 36 | LV-36 | R | K | L | L | X2 | K | L | K | X2 | K | R |
| 37 | LV-37 | R | K | L | L | X3 | K | L | K | X3 | K | R |
| 38 | LV-38 | R | K | L | L | X4 | K | L | K | X4 | K | R |
| 39 | LV-39 | R | K | L | L | X5 | K | L | K | X5 | K | R |
| 40 | LV-40 | K | K | L | L | X6 | R | L | K | X6 | R | K |
| 41 | LV-41 | R | K | L | L | X7 | K | L | K | X7 | K | R |
| 42 | LV-42 | R | K | X8 | X8 | X | R | X8 | K | X | R | K |
| 43 | LV-43 | R | K | X9 | X9 | X | R | X9 | K | X | R | K |
| 44 | LV-44 | R | K | X10 | X10 | X | R | X10 | K | X | R | K |
| 45 | LV-45 | R | K | A | A | X | R | A | K | X | R | K |
| 46 | LV-46 | R | K | V | V | X | R | V | K | X | R | K |
| 47 | LV-47 | K | K | L | L | X | K | L | K | X | K | K |
| 48 | LV-48 | R | K | L | L | X | K | L | K | X | K | R |
| 49 | LV-49 | K | K | L | L | X | R | L | K | X | R | K |
| 50 | LV-50 | R | K | L | L | X | R | L | K | X | R | K |
| 51 | LV-51 | R | R | L | L | X | R | L | K | X | R | K |
| 52 | LV-52 | R | K | L | L | X | R | L | K | X | R | R |
| 53 | LV-53 | K | R | L | L | X | K | L | K | X | K | R |
| 54 | LV-54 | R | R | L | L | X | K | L | K | X | K | R |
| 55 | LV-55 | K | K | L | L | X2 | R | L | K | X2 | R | K |
| | | |
|---|---|---|
| 56 | LV-56 | Ac-RKLLXRLKXRK-PEG200 |
| 57 | LV-57 | Ac-RKLLXRLKXRK-PEG500 |
| 58 | LV-58 | Ac-RKLLXRLKXRK-PEG1000 |
| 59 | LV-59 | PEG2000-RKLLXRLKXRK-NH₂ |
| 60 | LV-60 | Ac-RKLLXRLKXRK-PEG4000 |

wherein X represents a 3-(2-naphthyl)-alanine residue, X1 represents a 4-fluorophenylalanine residue, X2 represents a 3,4-difluorophenylalanine residue, X3 represents a 4-chlorophenylalanine residue, X4 represents a 3,4-dichlorophenylalanine residue, X5 represents a 4-(trifluoromethyl-)phenylalanine residue, X6 represents a 4-bromophenylalanine residue, X7 represents a 4-methyl-phenylalanine residue, X8 represents a norleucine residue, X9 represents a norvaline residue, X10 represents a homoleucine residue, Ac represents acetylation modification at an N-terminal of the polypeptide the antimicrobial peptide, and NH₂ represents amidation modification at a C-terminal of the polypeptide the antimicrobial peptide; and the phenylalanine-derived residue comprises the 4-fluorophenylalanine residue, the 3,4-difluorophenylalanine residue, the 4-chlorophenylalanine residue, the 3,4-dichlorophenylalanine residue, the 4-(trifluoromethyl-)phenylalanine residue, the 4-bromophenylalanine residue, and the 4-methyl-phenylalanine residue.

9. A pharmaceutical composition, comprising the AMP according to claim 1.

10. Application of the AMP according to claim 1 in the preparation of a drug for preventing and/or controlling microbial infection.

11. The application according to claim 10, wherein the drug comprises at least the AMPS according to claim 1, or a pharmaceutically acceptable salt, ester, a solvate, a hydrate or a prodrug thereof, and at least one pharmaceutically acceptable carrier, supplementary material, excipient, diluent, buffer, adjuvant, auxiliary agent, or vehicle.

12. The application according to claim 10, wherein a dosage form of the drug comprises an injectable formulation, an oral formulation, or a topical formulation.

13. The application according to claim 12, wherein the oral formulation comprises a tablet, an oral liquid formulation, a capsule, a granule or a traditional Chinese medicine formulation, and the topical formulation comprises an ointment, a gel, a suppository or an aerosol.

14. The application according to claim 10, comprising any of the following:
(1) application of the AMP in the preparation of anti-inflammatory and/or anti-tumor drugs;
(2) application of the AMP in aquaculture;
(3) application of the AMP in the preparation of oral care products;
(4) application of the AMP in the preparation of oral cleansing formulations;
(5) application of the AMP in the preparation of tooth cleaning products;
(6) application of the AMP in the preparation of dental coatings;
(7) application of the AMP in the preparation of drugs for the prevention and treatment of dental caries;
(8) application of the AMP in the preparation of products for treating skin infections;
(9) application of the AMP in the preparation of intracellular bactericidal products;
(10) application of the AMP in the preparation of products for removing bacterial biofilms;
(11) application of the AMP in the preparation of disinfectant products;
(12) application of the AMP in preventing and/or treating wheat Fusarium head blight;
(13) application of the AMP in the preparation of mildew-proof and preservative agents;
(14) application of the AMP in the preparation of food preservatives;
(15) application of the AMP in the preparation of cosmetic preservatives;
(16) application of the AMP in the preparation of cosmetics;
(17) application of the AMP in livestock and poultry feed and/or feed additives;
(18) application of the AMP in the improvement of animal and plant varieties and/or breeding of animal and plant;
(19) application of the AMP in the preparation of wound dressings;
(20) application of the AMP in the preparation of antimicrobial preservation films;
(21) application of the AMP in the preservation of fruit and vegetable;
(22) application of the AMP in livestock farming; and
(23) application of the AMP in the preparation of antimicrobial agents, pharmaceuticals, veterinary drugs, feeds, food products, daily chemicals, health care products, cosmetics, bactericides, virucides, algaecides, sterilants, additives, oral cleansing formulations, disinfectants, anti-inflammatory products, detergents, preservatives, supplementary materials, mildew-proof agents, algae-proof agents, detergent auxiliaries, detergent compositions, cleaning agents, or preservatives.

15. The antimicrobial peptide according to claim 2, wherein the AMP comprises all L-enantiomers or all D-enantiomers; or one or more amino acids in the common amino acid general formula are substituted with L-amino acids or D-amino acids.

16. The antimicrobial peptide according to claim 3, wherein the AMP comprises all L-enantiomers or all D-enantiomers; or one or more amino acids in the common amino acid general formula are substituted with L-amino acids or D-amino acids.

17. The antimicrobial peptide according to claim 4, wherein the AMP comprises all L-enantiomers or all D-enantiomers; or one or more amino acids in the common amino acid general formula are substituted with L-amino acids or D-amino acids.

18. The antimicrobial peptide according to claim 2, wherein polyethylene glycol (PEG) is conjugated to the N-terminal or the C-terminal of the AMP, or a side-chain amino group of the lys residue.

19. The antimicrobial peptide according to claim 3, wherein polyethylene glycol (PEG) is conjugated to the N-terminal or the C-terminal of the AMP, or a side-chain amino group of the lys residue.

20. The antimicrobial peptide according to claim 4, wherein polyethylene glycol (PEG) is conjugated to the N-terminal or the C-terminal of the AMP, or a side-chain amino group of the lys residue.


SEQUENCE LISTING (60 sequences)

SEQ ID NO: 1 - protein, 11 aa, unidentified
  MOD_RES 5: note = 3-(2- naphthyl)-alanine residue
  MOD_RES 9: note = 3-(2- naphthyl)-alanine residue
RRLLXRLRXRR

SEQ ID NO: 2 - protein, 11 aa, unidentified
RKLLFRLKFRK

SEQ ID NO: 3 - protein, 11 aa, unidentified
  MOD_RES 9: note = 3-(2- naphthyl)-alanine residue
RKLLFRLKXRK

SEQ ID NO: 4 - protein, 11 aa, unidentified
  MOD_RES 5: note = 3-(2-naphthyl)-alanine residue
RKLLXRLKFRK

SEQ ID NO: 5 - protein, 11 aa, unidentified
  MOD_RES 5: note = 4- fluoro-phenylalanine residue
  MOD_RES 9: note = 4- fluoro-phenylalanine residue
RKLLXKLKXKR

SEQ ID NO: 6 - protein, 11 aa, unidentified
  MOD_RES 5: note = 3,4-difluorophenylalanine residue
  MOD_RES 9: note = 3,4-difluorophenylalanine residue
RKLLXKLKXKR

SEQ ID NO: 7 - protein, 11 aa, unidentified
  MOD_RES 5: note = 4- chlorophenylalanine residue
  MOD_RES 9: note = 4- chlorophenylalanine residue
RKLLXKLKXKR

SEQ ID NO: 8 - protein, 11 aa, unidentified
  MOD_RES 5: note = 3,4- dichlorophenylalanine residue
  MOD_RES 9: note = 3,4- dichlorophenylalanine residue
RKLLXKLKXKR

SEQ ID NO: 9 - protein, 11 aa, unidentified
  MOD_RES 5: note = 4- trifluoromethyl-phenylalanine residue
  MOD_RES 9: note = 4- trifluoromethyl-phenylalanine residue
RKLLXKLKXKR

SEQ ID NO: 10 - protein, 11 aa, unidentified
  MOD_RES 5: note = 4- bromophenylalanine residue
  MOD_RES 9: note = 4- bromophenylalanine residue
KKLLXRLKXRK

SEQ ID NO: 11 - protein, 11 aa, unidentified
  MOD_RES 5: note = 4- methyl-phenylalanine residue
  MOD_RES 9: note = 4- methyl-phenylalanine residue
RKLLXKLKXKR

SEQ ID NO: 12 - protein, 11 aa, unidentified
  MOD_RES 3: note = norleucine residue
  MOD_RES 4: note = norleucine residue
  MOD_RES 7: note = norleucine residue
  MOD_RES 5: note = 3-(2- naphthyl)-alanine residue
  MOD_RES 9: note = 3-(2- naphthyl)-alanine residue
RKXXXRXKXRK

SEQ ID NO: 13 - protein, 11 aa, unidentified
  MOD_RES 3: note = norvaline residue
  MOD_RES 4: note = norvaline residue
  MOD_RES 7: note = norvaline residue
  MOD_RES 5: note = 3-(2- naphthyl)-alanine residue
  MOD_RES 9: note = 3-(2- naphthyl)-alanine residue
RKXXXRXKXRK

SEQ ID NO: 14 - protein, 11 aa, unidentified
  MOD_RES 3: note = Homoleucine residue
  MOD_RES 4: note = Homoleucine residue
  MOD_RES 7: note = Homoleucine residue
  MOD_RES 5: note = 3-(2- naphthyl)-alanine residue
  MOD_RES 9: note = 3-(2- naphthyl)-alanine residue
RKXXXRXKXRK

SEQ ID NO: 15 - protein, 11 aa, unidentified
  MOD_RES 5: note = 3-(2- naphthyl)-alanine residue
  MOD_RES 9: note = 3-(2- naphthyl)-alanine residue
RKAAXRAKXRK

SEQ ID NO: 16 - protein, 11 aa, unidentified
  MOD_RES 5: note = 3-(2- naphthyl)-alanine residue
  MOD_RES 9: note = 3-(2- naphthyl)-alanine residue
RKVVXRVKXRK

SEQ ID NO: 17 - protein, 11 aa, unidentified
  MOD_RES 5: note = 3-(2- naphthyl)-alanine residue
  MOD_RES 9: note = 3-(2- naphthyl)-alanine residue
KKLLXKLKXKK

SEQ ID NO: 18 - protein, 11 aa, unidentified
  MOD_RES 5: note = 3-(2- naphthyl)-alanine residue
  MOD_RES 9: note = 3-(2- naphthyl)-alanine residue
RKLLXKLKXKR

SEQ ID NO: 19 - protein, 11 aa, unidentified
  MOD_RES 5: note = 3-(2- naphthyl)-alanine residue
  MOD_RES 9: note = 3-(2- naphthyl)-alanine residue
KKLLXRLKXRK

SEQ ID NO: 20 - protein, 11 aa, unidentified
  MOD_RES 5: note = 3-(2- naphthyl)-alanine residue
  MOD_RES 9: note = 3-(2- naphthyl)-alanine residue
RKLLXRLKXRK

SEQ ID NO: 21 - protein, 11 aa, unidentified
  MOD_RES 5: note = 3-(2- naphthyl)-alanine residue
  MOD_RES 9: note = 3-(2- naphthyl)-alanine residue
RRLLXRLKXRK

SEQ ID NO: 22 - protein, 11 aa, unidentified
  MOD_RES 5: note = 3-(2- naphthyl)-alanine residue
  MOD_RES 9: note = 3-(2- naphthyl)-alanine residue
RKLLXRLKXRR

SEQ ID NO: 23 - protein, 11 aa, unidentified
  MOD_RES 5: note = 3-(2- naphthyl)-alanine residue
  MOD_RES 9: note = 3-(2- naphthyl)-alanine residue
KRLLXKLKXKR

SEQ ID NO: 24 - protein, 11 aa, unidentified
  MOD_RES 5: note = 3-(2- naphthyl)-alanine residue
  MOD_RES 9: note = 3-(2- naphthyl)-alanine residue
RRLLXKLKXKR

SEQ ID NO: 25 - protein, 11 aa, unidentified
  MOD_RES 5: note = 3,4-difluorophenylalanine residue
  MOD_RES 9: note = 3,4-difluorophenylalanine residue
KKLLXRLKXRK

SEQ ID NO: 26 - protein, 11 aa, unidentified
  MOD_RES 5: note = 3-(2- naphthyl)-alanine residue
  MOD_RES 9: note = 3-(2- naphthyl)-alanine residue
RKLLXRLKXRK

SEQ ID NO: 27 - protein, 12 aa, unidentified
  MOD_RES 5: note = 3-(2- naphthyl)-alanine residue
  MOD_RES 9: note = 3-(2- naphthyl)-alanine residue
RKLLXRLKXRKK

SEQ ID NO: 28 - protein, 12 aa, unidentified
  MOD_RES 6: note = 3-(2- naphthyl)-alanine residue
  MOD_RES 10: note = 3-(2- naphthyl)-alanine residue
KRKLLXRLKXRK

SEQ ID NO: 29 - protein, 11 aa, unidentified
  MOD_RES 5: note = 3-(2- naphthyl)-alanine residue
  MOD_RES 9: note = 3-(2- naphthyl)-alanine residue
RKLLXRLKXRK

SEQ ID NO: 30 - protein, 11 aa, unidentified
  MOD_RES 5: note = 3-(2- naphthyl)-alanine residue
  MOD_RES 9: note = 3-(2- naphthyl)-alanine residue
RKLLXRLKXRK

SEQ ID NO: 31 - protein, 11 aa, unidentified
  MOD_RES 5: note = 3-(2- naphthyl)-alanine residue
  MOD_RES 9: note = 3-(2- naphthyl)-alanine residue
RRLLXRLRXRR

SEQ ID NO: 32 - protein, 11 aa, unidentified
RKLLFRLKFRK

SEQ ID NO: 33 - protein, 11 aa, unidentified
  MOD_RES 9: note = 3-(2-naphthyl)-alanine residue
RKLLFRLKXRK

SEQ ID NO: 34 - protein, 11 aa, unidentified
  MOD_RES 5: note = 3-(2- naphthyl)-alanine residue
RKLLXRLKFRK

SEQ ID NO: 35 - protein, 11 aa, unidentified
  MOD_RES 5: note = 4- fluoro-phenylalanine residue
  MOD_RES 9: note = 4- fluoro-phenylalanine residue
RKLLXKLKXKR

SEQ ID NO: 36 - protein, 11 aa, unidentified
  MOD_RES 5: note = 3,4-difluorophenylalanine residue
  MOD_RES 9: note = 3,4-difluorophenylalanine residue
RKLLXKLKXKR

SEQ ID NO: 37 - protein, 11 aa, unidentified
  MOD_RES 5: note = 4- chlorophenylalanine residue
  MOD_RES 9: note = 4- chlorophenylalanine residue
RKLLXKLKXKR

SEQ ID NO: 38 - protein, 11 aa, unidentified
  MOD_RES 5: note = 3,4- dichlorophenylalanine residue
  MOD_RES 9: note = 3,4- dichlorophenylalanine residue
RKLLXKLKXKR

SEQ ID NO: 39 - protein, 11 aa, unidentified
  MOD_RES 5: note = 4- trifluoromethyl-phenylalanine residue
  MOD_RES 9: note = 4- trifluoromethyl-phenylalanine residue
RKLLXKLKXKR

SEQ ID NO: 40 - protein, 11 aa, unidentified
  MOD_RES 5: note = 4- bromophenylalanine residue
  MOD_RES 9: note = 4- bromophenylalanine residue
KKLLXRLKXRK

SEQ ID NO: 41 - protein, 11 aa, unidentified
  MOD_RES 5: note = 4- methyl-phenylalanine residue
  MOD_RES 9: note = 4- methyl-phenylalanine residue
RKLLXKLKXKR

SEQ ID NO: 42 - protein, 11 aa, unidentified
  MOD_RES 3: note = norleucine residue
  MOD_RES 4: note = norleucine residue
  MOD_RES 7: note = norleucine residue
  MOD_RES 5: note = 3-(2- naphthyl)-alanine residue
  MOD_RES 9: note = 3-(2- naphthyl)-alanine residue
RKXXXRXKXRK

SEQ ID NO: 43 - protein, 11 aa, unidentified
  MOD_RES 5: note = 3-(2- naphthyl)-alanine residue
  MOD_RES 9: note = 3-(2- naphthyl)-alanine residue
  MOD_RES 3: note = norvaline residue
  MOD_RES 4: note = norvaline residue
  MOD_RES 7: note = norvaline residue
RKXXXRXKXRK

SEQ ID NO: 44 - protein, 11 aa, unidentified
  MOD_RES 3: note = Homoleucine residue
  MOD_RES 4: note = Homoleucine residue
  MOD_RES 7: note = Homoleucine residue
  MOD_RES 5: note = 3-(2- naphthyl)-alanine residue
  MOD_RES 9: note = 3-(2- naphthyl)-alanine residue
RKXXXRXKXRK

SEQ ID NO: 45 - protein, 11 aa, unidentified
  MOD_RES 5: note = 3-(2- naphthyl)-alanine residue
  MOD_RES 9: note = 3-(2- naphthyl)-alanine residue
RKAAXRAKXRK

SEQ ID NO: 46 - protein, 11 aa, unidentified
  MOD_RES 5: note = 3-(2- naphthyl)-alanine residue
  MOD_RES 9: note = 3-(2- naphthyl)-alanine residue
RKVVXRVKXRK

SEQ ID NO: 47 - protein, 11 aa, unidentified
  MOD_RES 5: note = 3-(2- naphthyl)-alanine residue
  MOD_RES 9: note = 3-(2- naphthyl)-alanine residue
KKLLXKLKXKK

SEQ ID NO: 48 - protein, 11 aa, unidentified
  MOD_RES 5: note = 3-(2- naphthyl)-alanine residue
  MOD_RES 9: note = 3-(2- naphthyl)-alanine residue
RKLLXKLKXKR

SEQ ID NO: 49 - protein, 11 aa, unidentified
  MOD_RES 5: note = 3-(2- naphthyl)-alanine residue
  MOD_RES 9: note = 3-(2- naphthyl)-alanine residue
KKLLXRLKXRK

SEQ ID NO: 50 - protein, 11 aa, unidentified
  MOD_RES 5: note = 3-(2- naphthyl)-alanine residue
  MOD_RES 9: note = 3-(2- naphthyl)-alanine residue
RKLLXRLKXRK

SEQ ID NO: 51 - protein, 11 aa, unidentified
  MOD_RES 5: note = 3-(2- naphthyl)-alanine residue
  MOD_RES 9: note = 3-(2- naphthyl)-alanine residue
RRLLXRLKXRK

SEQ ID NO: 52 - protein, 11 aa, unidentified
  MOD_RES 5: note = 3-(2- naphthyl)-alanine residue
  MOD_RES 9: note = 3-(2- naphthyl)-alanine residue
RKLLXRLKXRR

SEQ ID NO: 53 - protein, 11 aa, unidentified
  MOD_RES 5: note = 3-(2- naphthyl)-alanine residue
  MOD_RES 9: note = 3-(2- naphthyl)-alanine residue
KRLLXKLKXKR

SEQ ID NO: 54 - protein, 11 aa, unidentified
  MOD_RES 5: note = 3-(2- naphthyl)-alanine residue
  MOD_RES 9: note = 3-(2- naphthyl)-alanine residue
RRLLXKLKXKR

SEQ ID NO: 55 - protein, 11 aa, unidentified
  MOD_RES 5: note = 3,4-difluorophenylalanine residue
  MOD_RES 9: note = 3,4-difluorophenylalanine residue
KKLLXRLKXRK

SEQ ID NO: 56 - protein, 11 aa, unidentified
  MOD_RES 5: note = 3-(2- naphthyl)-alanine residue
  MOD_RES 9: note = 3-(2- naphthyl)-alanine residue
RKLLXRLKXRK

SEQ ID NO: 57 - protein, 12 aa, unidentified
  MOD_RES 5: note = 3-(2- naphthyl)-alanine residue
  MOD_RES 9: note = 3-(2- naphthyl)-alanine residue
RKLLXRLKXRKK

SEQ ID NO: 58 - protein, 12 aa, unidentified
  MOD_RES 6: note = 3-(2- naphthyl)-alanine residue
  MOD_RES 10: note = 3-(2- naphthyl)-alanine residue
KRKLLXRLKXRK

SEQ ID NO: 59 - protein, 11 aa, unidentified
  MOD_RES 5: note = 3-(2- naphthyl)-alanine residue
  MOD_RES 9: note = 3-(2- naphthyl)-alanine residue
RKLLXRLKXRK

SEQ ID NO: 60 - protein, 11 aa, unidentified
  MOD_RES 5: note = 3-(2- naphthyl)-alanine residue
  MOD_RES 9: note = 3-(2- naphthyl)-alanine residue
RKLLXRLKXRK